(19) United States
(12) Patent Application Publication
(10) Pub. No.: US 2026/0269067 A1
(43) Pub. Date: Sep. 10, 2026

(54) METHOD FOR DIAGNOSING CARDIOVASCULAR DISEASE AND DEVICE USING THE SAME

(71) Applicant: MEDIWHALE INC., Seoul (KR)

(72) Inventors: Tae Geun CHOI, Seoul (KR); Hyung Taek RIM, Seoul (KR); Geun Yeong LEE, Seoul (KR)

(21) Appl. No.: 19/662,909

(22) Filed: Apr. 29, 2026

Related U.S. Application Data
(63) Continuation of application No. PCT/KR2023/016949, filed on Oct. 29, 2023.

(30) Foreign Application Priority Data
Oct. 29, 2023 (KR) 10-2023-0146159

(51) Int. Cl. G16H 50/20

(52) U.S. Cl. CPC G16H 50/20

(57) ABSTRACT

Disclosed are a method for diagnosing cardiovascular disease and a device using the same. A control method of a diagnostic device according to one embodiment may comprise: obtaining a retinal image of a subject; and obtaining cardiovascular disease diagnostic information about the subject by using a machine learning model based on the retinal image, wherein the machine learning model includes a first model and a second model, wherein the first model is a neural network model, and wherein the second model may be a regression-based machine learning model.

### CROSS-REFERENCE TO RELATED APPLICATIONS

This application is a continuation of International Application No. PCT/KR2023/016949, filed on Oct. 29, 2023, which claims priority to and the benefit of Korean Patent Application No. 10-2023-0146159, filed on Oct. 29, 2023. The entire contents of the above-referenced applications are incorporated herein by reference in their entirety.

### TECHNICAL FIELD

The present application relates to a method for diagnosing cardiovascular disease and a device using the same.

### BACKGROUND

Retinal examination is a diagnostic material frequently used in ophthalmology because it can observe abnormalities of the retina, optic nerve, and macular region, and the results can be confirmed relatively simply through imaging. Meanwhile, due to the rapid advancement of artificial intelligence technology in recent years, the development of diagnostic artificial intelligence in the medical diagnosis field, particularly in the image-based diagnosis field, is being actively carried out. Global companies are also not sparing investment in artificial intelligence development for analyzing various medical imaging data, including by inputting large-scale data through cooperation with the medical community, and some companies have succeeded in developing artificial intelligence diagnostic tools that output excellent diagnostic results.

Since retinal images allow non-invasive observation of blood vessels within the body, there is a demand to expand the application of diagnosis using retinal images not only for ophthalmic diseases but also in relation to cardiovascular diseases.

### SUMMARY

A technical object of the present application is to provide a method for diagnosing cardiovascular disease, which obtains information about cardiovascular disease with high accuracy using a retinal image and a machine learning model.

The technical objects of the present application are not limited to the above-mentioned objects, and the objects not mentioned can be clearly understood by those skilled in the art to which the present application pertains from the present specification and the attached drawings.

According to one aspect, a control method of a diagnostic device according to one embodiment may comprise: obtaining a retinal image of a subject; and obtaining cardiovascular disease diagnostic information about the subject by using a machine learning model based on the retinal image, wherein the machine learning model includes a first model and a second model, wherein the first model is a neural network model, and wherein the second model may be a regression-based machine learning model.

The technical solutions are not limited to the above-mentioned solving means, and technical solutions not mentioned can be clearly understood by those skilled in the art to which the present application pertains from the present specification and the attached drawings.

According to the present application, information about cardiovascular disease can be obtained with high accuracy using a retinal image and a machine learning model.

The advantageous effects of the invention of the present application are not limited to the above-mentioned effects, and effects not mentioned can be clearly understood by those skilled in the art to which the present application pertains from the present specification and the attached drawings.

### BRIEF DESCRIPTION OF THE DRAWINGS

FIG. 1 illustrates a diagnostic system according to one embodiment.

FIG. 2 is a block diagram for explaining a learning device according to one embodiment.

FIG. 3 is a block diagram for explaining a diagnostic device according to one embodiment.

FIG. 4 illustrates a diagnostic system according to one embodiment.

FIG. 5 is a block diagram for explaining a client device according to one embodiment.

FIG. 6 is a diagram for explaining a diagnostic process according to one embodiment.

FIG. 7 is a diagram for explaining a configuration of a learning unit according to one embodiment.

FIG. 8 is a conceptual diagram for explaining an image data set according to one embodiment.

FIG. 9 is a block diagram for explaining a learning process of a diagnostic model according to one embodiment.

FIG. 10 is a diagram for explaining a configuration of a diagnostic unit according to one embodiment.

FIG. 11 is a diagram for explaining a diagnostic process according to one embodiment.

FIG. 12 is a block diagram for explaining a diagnostic unit according to one embodiment.

FIG. 13 is a diagram for explaining a diagnostic process according to one embodiment.

FIG. 14 is a diagram for explaining a diagnostic system according to one embodiment.

FIG. 15 is a diagram for explaining a serial diagnostic model according to one embodiment.

FIG. 16 is a diagram for explaining a serial diagnostic model according to another embodiment.

FIG. 17 is a diagram for explaining a serial diagnostic model according to yet another embodiment.

FIG. 18 is a diagram for explaining a diagnostic method using a diagnostic model according to one embodiment.

FIG. 19 is a diagram for explaining a method for diagnosing cardiovascular disease according to one embodiment.

FIG. 20 illustrates a diagnostic model for obtaining cardiovascular disease diagnostic information according to one embodiment.

FIG. 21 shows clinical characteristics of subjects according to cardiovascular disease diagnostic information according to Example 1.

FIGS. 22A and 22B are diagrams for explaining incidence rates of cardiovascular disease-related events according to cardiovascular disease diagnostic information and QRISK3 according to Example 1.

FIG. 23 is a diagram for explaining prediction performance of occurrence of cardiovascular disease-related events within 10 years of cardiovascular disease diagnostic information according to Example 1.

FIGS. 24A to 24C are diagrams for explaining results of applying cardiovascular disease diagnostic information to a group having a QRISK3 score of 7.5 to 10% according to Example 1.

FIG. 25 is a diagram for explaining in detail performance when cardiovascular disease diagnostic information and a QRISK3 score are applied together according to Example 1.

FIG. 26 shows clinical characteristics of subjects according to cardiovascular disease diagnostic information according to Example 2.

FIG. 27 is a diagram for explaining performance of cardiovascular disease diagnostic information according to Example 2.

FIG. 28 is a diagram for explaining performance of cardiovascular disease diagnostic information with respect to various ethnicities according to Example 2.

FIG. 29 is a diagram for explaining a method for diagnosing cardiovascular disease according to another embodiment.

FIG. 30 is a diagram for explaining a method for providing guide information for cardiovascular disease diagnostic information according to one embodiment.

FIG. 31 is a diagram for explaining a method for providing guide information using a result according to an existing biomarker and cardiovascular disease diagnostic information according to one embodiment.

FIG. 32 is a diagram for explaining a method for predicting a cardiovascular disease progression speed using cardiovascular disease diagnostic information according to one embodiment.

A control method of a diagnostic device according to one embodiment may comprise: obtaining a retinal image of a subject; and obtaining cardiovascular disease diagnostic information about the subject by using a machine learning model based on the retinal image, wherein the machine learning model includes a first model and a second model, wherein the first model is a neural network model, and wherein the second model may be a regression-based machine learning model.

### DETAILED DESCRIPTION

The above-mentioned objects, features, and advantages of the present application will become more apparent through the following detailed description related to the attached drawings. However, since the present application can have various changes and can have various embodiments, specific embodiments will be illustrated in the drawings and described in detail below.

In the drawings, the thicknesses of layers and regions are exaggerated for clarity, and also, when an element or layer is referred to as being “on” or “above” another element or layer, it includes not only the case where it is directly on the other element or layer but also the case where another layer or another element is interposed therebetween. Throughout the specification, the same reference numerals indicate, in principle, the same elements. In addition, elements having the same function within the scope of the same idea appearing in the drawings of each embodiment are described using the same reference numerals.

When it is determined that a detailed description of a known function or configuration related to the present application may unnecessarily obscure the gist of the present application, the detailed description thereof will be omitted. In addition, numerals (for example, first, second, etc.) used in the description process of the present specification are merely identification symbols for distinguishing one element from another element.

In addition, the suffixes “module” and “unit” for elements used in the following description are given or used interchangeably only in consideration of ease of writing the specification, and do not have meanings or roles that are distinguished from each other per se.

The method according to the embodiment may be implemented in the form of program instructions that can be executed through various computer means and recorded on a computer-readable medium. The computer-readable medium may include program instructions, data files, data structures, etc., alone or in combination. The program instructions recorded on the medium may be specially designed and configured for the embodiment or may be known and available to those skilled in computer software. Examples of computer-readable recording media include magnetic media such as hard disks, floppy disks, and magnetic tapes, optical media such as CD-ROMs and DVDs, magneto-optical media such as floptical disks, and hardware devices specially configured to store and execute program instructions such as ROM, RAM, flash memory, etc. Examples of program instructions include not only machine language code such as that created by a compiler but also high-level language code that can be executed by a computer using an interpreter, etc. The above-described hardware devices may be configured to operate as one or more software modules to perform the operations of the embodiments, and vice versa.

### 1. DIAGNOSIS USING RETINAL IMAGES

### 1.1. Diagnostic System and Process

### 1.1.1. Purpose and Definitions

Hereinafter, a diagnostic system and method for assisting medical staff's judgment regarding the presence or absence of disease or abnormality that serves as a basis for such judgment based on ocular images will be described. In the present specification, the term diagnosis may mean diagnostic assistance for assisting in the diagnosis of disease rather than directly diagnosing disease. Hereinafter, the term diagnosis is used for convenience of description, but this may mean diagnostic assistance.

In particular, a diagnostic method for constructing a machine learning model for diagnosing disease using deep learning techniques and assisting in the detection of the presence or absence of disease or abnormal findings using the constructed model will be described. In the present specification, an ocular image is an image including the eye of a subject, and may include various images such as a retinal image and/or a fundus image. In the present specification, the technology of the present specification is described focusing on retinal images for convenience of description, but is not limited thereto, and it goes without saying that the description of the present specification can also be applied to other ocular images.

The machine learning model described in the present specification may be designed based on various machine learning libraries. For example, the machine learning model may mean various types of models designed based on supervised, unsupervised, semi-supervised, or reinforcement learning artificial intelligence algorithms such as decision tree, random forest algorithm, stochastic gradient descent algorithm, neural network algorithm, k-nearest neighbor algorithm, linear regression, logistic regression, Cox proportional hazards model (survival model) (regression-based), support vector machine, k-means, Hierarchical Cluster Analysis (HCA), Expectation Maximization, Principal Component Analysis (PCA), Kernel PCA, Locally-Linear Embedding (LLE), t-distributed Stochastic Neighbor Embedding (t-SNE), Apriori, and Eclat.

Hereinafter, unless otherwise specifically described, the machine learning model is mainly described as a neural network model for convenience, but this does not necessarily mean only a model form based on a neural network algorithm, and it is obvious that it can be replaced with a model based on another algorithm within the functional and purpose scope of the invention described in the present specification.

According to one embodiment, a diagnostic system or method for assisting in the diagnosis of at least one of ophthalmic disease, cardiovascular disease (and/or cardio-cerebrovascular disease), renal disease, and other systemic diseases based on retinal images may be provided.

Illustratively, in the present specification, ophthalmic disease may include at least one of Cataract, Glaucoma, Macular Degeneration, Diabetic Retinopathy, Epiretinal membrane, macular hole, high/degenerative myopia, melanoma, Retinal Detachment, Dry Eye Syndrome, Presbyopia, and Astigmatism.

In addition, cardiovascular disease may include at least one of Coronary Artery Disease (CAD), aortic valve stenosis, Hypertension, Arterial hypertension, Heart Failure, arrhythmia, Atrial Fibrillation, Valvular Heart Disease, Cardiomyopathy, Peripheral vascular disease, Peripheral Artery Disease (PAD), Heart Attack, Aneurysm, aortic aneurysm (for example, abdominal aortic aneurysm, thoracic aortic aneurysm), thrombotic disease (for example, deep vein thrombosis, pulmonary embolism), myocardial infarction, and cardio-cerebrovascular disease. In addition, cardio-cerebrovascular disease may include at least one of Stroke, ischemic stroke, cerebral infarction, cerebral hemorrhage, subarachnoid hemorrhage, transient ischemic attack, and death due to cardiovascular disease. Cardiovascular disease may include complications. In addition, complications may include aspiration pneumonia, dysphagia, decreased motor function, decreased language function, decreased cognitive function, sleep disorder, emotional disorder, cranial neuropathic pain, urinary tract infection, malnutrition, deep vein thrombosis, pressure ulcer, fall, pain, seizure, depression, etc.

In addition, renal disease may include at least one of Chronic Kidney Disease (CKD), Acute Kidney Injury (AKI), Kidney Stones, Nephrotic Syndrome, Glomerulonephritis, Polycystic Kidney Disease (PKD), Kidney Cancer, and Pyelonephritis. In addition, renal disease may include complications. In addition, complications may include side effects due to dialysis, hypotension, muscle cramps, nausea and vomiting, headache, dialysis disequilibrium syndrome, pruritus, arrhythmia, etc.

In addition, other systemic diseases may include at least one of diabetes, hypertension, hypotension, Alzheimer's disease, cytomegalovirus, and arteriosclerosis.

In addition, according to another embodiment, various parameters of a subject can be predicted based on retinal images. For example, the parameters may include at least one of parameters representing physical information of the subject such as biological age, sex, height, weight, BMI index, body mass, body fat percentage, body muscle mass, etc. In addition, the parameters may include at least one of diagnostic value parameters such as hematocrit level, red blood cell count, white blood cell count, hemoglobin level, platelet count, total iron binding capacity (TIBC), iron level, ferritin (storage iron protein) level, total protein level, albumin level, aspartate transaminase level (AST), amino transaminase level, γ-GTP, γ-GT, alkaline phosphatase (ALP) level, globulin level, hepatitis antigen level, hepatitis antibody level, glycated hemoglobin level (HbA1C), blood urea nitrogen (BUN) level, creatinine level, uric acid level, total cholesterol level, high-density lipoprotein (HDL Cholesterol) level, low-density lipoprotein (LDL Cholesterol) level, triglyceride (TG) level, bicarbonate level, systolic blood pressure (SBP), diastolic blood pressure (DBP), etc.

According to yet another embodiment, a diagnostic system or method for detecting abnormal retinal findings that can be used in the diagnosis of ophthalmic diseases or other diseases may be provided. For example, a diagnostic system or method for obtaining finding information such as color abnormality of the entire retina, Opacity, abnormality of cup to disc ratio (C/D ratio) of the optic disc, macular abnormality (for example, macular hole), abnormality of vessel diameter, course, etc., retinal artery diameter abnormality, retinal hemorrhage, microaneurysm, hard exudate, Epiretinal membrane, myelinated nerve fiber, chorioretinal atrophy, RNFL defect, occurrence of exudate, drusen, cataract, glaucoma, diabetic retinopathy, Tessellated fundus, Large optic cup, retinal vein occlusion (RVO), branch retinal vein occlusion (BRVO), central retinal vein occlusion (CRVO), retinal artery occlusion (RAO), Rhegmatogenous RD, Posterior serous/exudative RD, central serous chorioretinopathy (CSCR), VKH disease, Maculopathy, epiretinal membrane (ERM), Macular Hole (MH), Pathological myopia, Optic nerve degeneration, Optic atrophy, Severe hypertensive retinopathy, Disc swelling and elevation, Dragged disc, Pigmentary degeneration, Congenital disc abnormality, Retinitis pigmentosa, Bietti crystalline dystrophy, Peripheral retinal degeneration and break, Myelinated nerve fiber, Vitreous particles, Fundus neoplasm, Massive hard exudates, Yellow-white spots/flecks, Cotton-wool spots, Vessel tortuosity, Chorioretinal atrophy/coloboma, Preretinal haemorrhage, Fibrosis, Laser spots, Silicon oil in eye, Blur fundus, Blur fundus without PDR, Blur fundus with suspected PDR, etc., may be provided.

In the present specification, diagnostic information may be understood as encompassing diagnostic information according to the determination of the presence or absence of disease or finding information that serves as the basis thereof.

### 1.1.2. Diagnostic System Configuration

According to one embodiment, a diagnostic system may be provided.

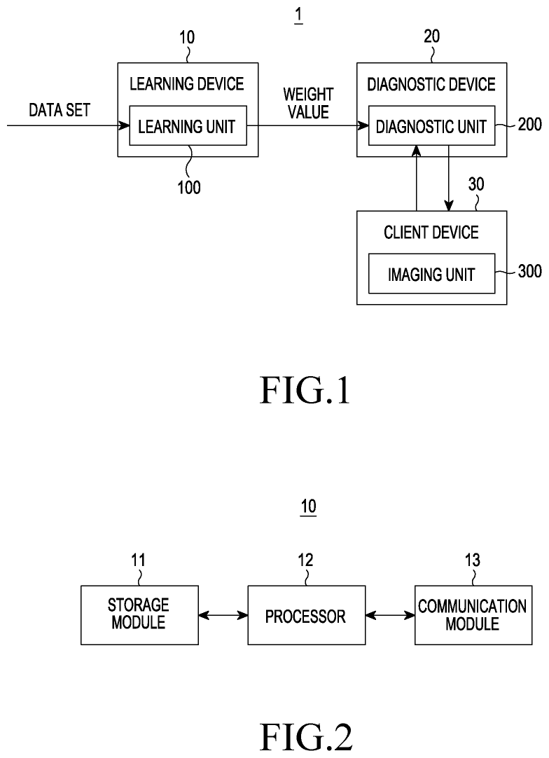

FIG. 1 illustrates a diagnostic system according to one embodiment. Referring to FIG. 1, the diagnostic system (1) may include a learning device (10) that trains a diagnostic model, a diagnostic device (20) that performs diagnosis using the diagnostic model, and a client device (30) that obtains a diagnosis request. The diagnostic system (1) may include a plurality of learning devices, a plurality of diagnostic devices, or a plurality of client devices.

The learning device (10) may include a learning unit (100). The learning unit (100) may perform training of a diagnostic model. As an example, the learning unit (100) may obtain a retinal image data set and perform training of a diagnostic model that detects disease or abnormal findings from retinal images. The learning unit (100) is included in a processor of the learning device (10) to be described later, and may mean that the processing for learning of the processor is functionally represented.

The diagnostic device (20) may include a diagnostic unit (200). The diagnostic unit (200) may perform diagnosis of disease or acquisition of assistant information used for diagnosis using a diagnostic model. As an example, the diagnostic unit (200) may perform acquisition of diagnostic information using the diagnostic model trained by the learning unit. The diagnostic unit (200) is included in a processor of the diagnostic device (20) to be described later, and may mean that the processing for diagnosis of the processor is functionally represented.

The client device (30) may include an imaging unit (300). The imaging unit (300) may image a retinal image. The client device may be an ophthalmic retinal imaging device. Alternatively, the client device (30) may be a handheld device such as a smartphone or a tablet PC.

In the diagnostic system (1) according to the present embodiment, the learning device (10) obtains a data set and performs learning of a diagnostic model to determine a diagnostic model for use in diagnosis, the diagnostic device obtains diagnostic information according to a diagnostic target image using the determined diagnostic model when an information request is obtained from a client, and the client device may request information from the diagnostic device and obtain diagnostic information transmitted in response thereto.

A diagnostic system according to another embodiment may include a diagnostic device that performs learning of a diagnostic model and diagnosis using the same, and a client device. A diagnostic system according to yet another embodiment may include a diagnostic device that performs learning of a diagnostic model, acquisition of a diagnosis request, and diagnosis. A diagnostic system according to yet another embodiment may include a learning device that performs learning of a diagnostic model, and a diagnostic device that obtains a diagnosis request and performs diagnosis.

The diagnostic system disclosed in the present specification is not limited to the embodiments described above, and may be implemented in any form including a learning unit that performs learning of a model, a diagnostic unit that obtains diagnostic information according to the learned model, and an imaging unit that obtains a diagnostic target image.

Hereinafter, some embodiments of each device constituting the system will be described.

### 1.1.2.1. Learning Device

A learning device according to one embodiment may perform training of a diagnostic model that assists diagnosis.

FIG. 2 is a block diagram for explaining a learning device according to one embodiment. Referring to FIG. 2, the learning device (10) may include a processor (12) and a storage module (11).

The learning device (10) may include a processor (12). The processor (12) may control the operation of the learning device (10).

The processor (12) may include one or more of a Central Processing Unit (CPU), Random Access Memory (RAM), Graphic Processing Unit (GPU), one or more microprocessors, and other electronic components capable of processing input data according to predetermined logic. In addition, the processor (12) may be at least one or more.

The processor (12) may read system programs and various processing programs stored in the storage module (11). As an example, the processor (12) may develop processes, methods, etc. for performing diagnosis to be described later on RAM, and perform various processing according to the developed program. The processor (12) may perform learning of a diagnostic model to be described later.

The learning device (10) may include a storage module (11). The storage module (11) may store data and learning models necessary for learning.

The storage module (11) may be implemented as a non-volatile semiconductor memory, hard disk, flash memory, RAM, Read Only Memory (ROM), Electrically Erasable Programmable Read-Only Memory (EEPROM), or other tangible non-volatile recording media.

The storage module (11) may store various processing programs, parameters for performing processing of the programs, or data of such processing results. As an example, the storage module (11) may store a data processing process program for performing diagnosis to be described later, a diagnostic process program, parameters for performing each program, and data obtained by performing such programs (for example, processed data or diagnostic result values). In addition, the storage module (11) may store various diagnostic models to be described later.

The learning device (10) may include a separate learning unit. The learning unit may perform learning of a diagnostic model.

The learning unit may be included in the processor (12) described above. The learning unit may be stored in the storage module (11) described above. The learning unit may be implemented by some components of the processor (12) and the storage module (11) described above. For example, the learning unit may be stored in the storage module (11) and driven by the processor (12).

The learning device (10) may further include a communication module (13). The communication module (13) may perform communication with an external device. For example, the communication module (13) may communicate with a diagnostic device, server device, or client device to be described later. The communication module (13) may perform wired or wireless communication. The communication module (13) may perform bi-directional or unidirectional communication.

### 1.1.2.2. Diagnostic Device

The diagnostic device may obtain diagnostic information using a diagnostic model.

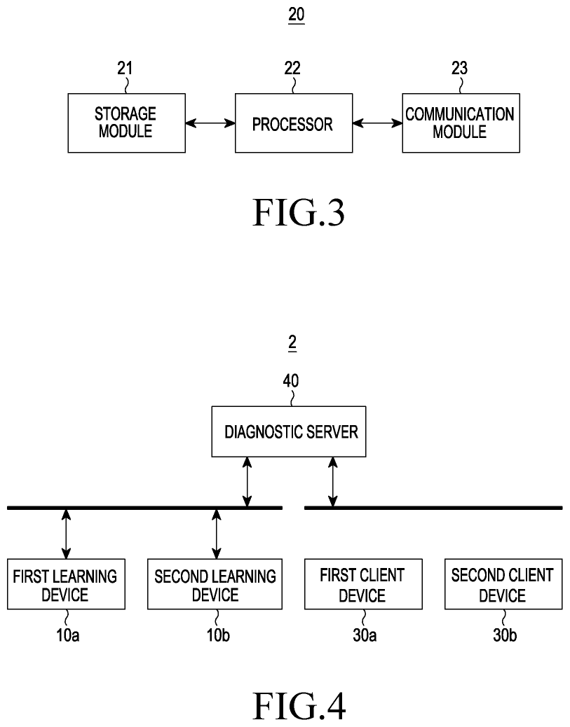

FIG. 3 is a block diagram for explaining a diagnostic device according to one embodiment. Referring to FIG. 3, the diagnostic device (20) may include a processor (22) and a storage module (21).

The processor (22) may include one or more of a Central Processing Unit (CPU), Random Access Memory (RAM), Graphic Processing Unit (GPU), one or more microprocessors, and other electronic components capable of processing input data according to predetermined logic. In addition, the processor (22) may be at least one or more.

The processor (22) may read system programs and various processing programs stored in the storage module (21). As an example, the processor (22) may develop processes, methods, etc. for performing diagnosis to be described later on RAM, and perform various processing according to the developed program. The processor (22) may generate diagnostic information using a diagnostic model. The processor (22) may obtain diagnostic data for diagnosis (for example, retinal data of a subject) and obtain diagnostic information predicted by the diagnostic data using a learned diagnostic model.

The storage module (21) may store a diagnostic model. The storage module (21) may store parameters, variables, etc. of the diagnostic model.

The storage module (21) may be implemented as a non-volatile semiconductor memory, hard disk, flash memory, RAM, Read Only Memory (ROM), Electrically Erasable Programmable Read-Only Memory (EEPROM), or other tangible non-volatile recording media.

The storage module (21) may store various processing programs, parameters for performing processing of the programs, or data of such processing results. As an example, the storage module (21) may store a data processing process program for performing diagnosis to be described later, a diagnostic process program, parameters for performing each program, and data obtained by performing such programs (for example, processed data or diagnostic result values). In addition, the storage module (21) may store various diagnostic models to be described later.

Although not shown, the diagnostic device (20) may further include an input module. The input module may obtain user input. For example, the input module may obtain user input requesting diagnostic information. In addition, the input module may obtain physical information of the subject (at least one of height, weight, age, sex, race, smoking status, blood pressure (for example, blood pressure level, presence or absence of hypertension), presence or absence of diabetes (or blood sugar level), cholesterol level). The processor (22) may obtain information input through the input module.

The diagnostic device (20) may further include a communication module (23). The communication module (23) may communicate with the learning device and/or the client device. As an example, the diagnostic device (20) may be provided in the form of a server that communicates with a client device. In this regard, it will be described in more detail below.

### 1.1.2.3. Server Device

According to one embodiment, the diagnostic system may include a server device. The diagnostic system according to one embodiment may include a plurality of server devices.

The server device may store and/or drive a diagnostic model. The server device may store weight values constituting a learned diagnostic model. The server device may collect or store data used for diagnosis.

The server device may output the result of a diagnostic process using a diagnostic model to a client device. The server device may obtain feedback from a client device. The server device may operate similarly to the diagnostic device described above.

FIG. 4 illustrates a diagnostic system according to one embodiment. Referring to FIG. 4, the diagnostic system (20) according to one embodiment may include a diagnostic server (40), a learning device, and a client device.

The diagnostic server (40), that is, the server device, may communicate with a plurality of learning devices or a plurality of diagnostic devices. Referring to FIG. 6, the diagnostic server (40) may communicate with a first learning device (10a) and a second learning device (10b). Referring to FIG. 4, the diagnostic server (40) may communicate with a first client device (30a) and a second client device (30b).

For example, the diagnostic server (40) may communicate with a first learning device (10a) that trains a first diagnostic model for obtaining first diagnostic information and a second learning device (10b) that trains a second diagnostic model for obtaining second diagnostic information.

The diagnostic server (40) may store a first diagnostic model for obtaining first diagnostic information and a second diagnostic model for obtaining second diagnostic information, obtain diagnostic information in response to a diagnostic information acquisition request from the first client device (30a) or the second client device (30b), and transmit the obtained diagnostic information to the first client device (30a) or the second client device (30b).

Alternatively, the diagnostic server (40) may communicate with a first client device (30a) that requests first diagnostic information and a second client device (30b) that requests second diagnostic information.

### 1.1.2.4. Client Device

The client device may request diagnostic information from a diagnostic device or a server device. The client device may obtain data necessary for diagnosis and transmit the obtained data to the diagnostic device.

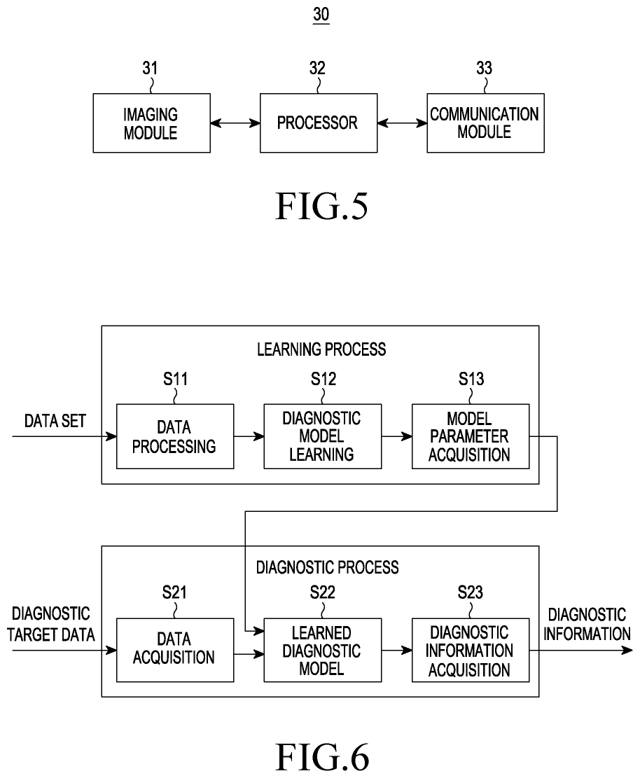

FIG. 5 is a block diagram for explaining a client device according to one embodiment. Referring to FIG. 5, the client device (30) according to one embodiment may include an imaging module (31), a processor (32), and a communication module (33).

The imaging module (31) may obtain image or video data. The imaging module (31) may obtain a retinal image. However, the client device (30) may be replaced with another form of data acquisition unit other than the imaging module (31).

The communication module (33) may communicate with an external device, such as a diagnostic device or a server device. The communication module (33) may perform wired or wireless communication.

The processor (32) may control the imaging module (31) to obtain an image or data. The processor (32) may control the imaging module (31) to obtain a retinal image. The processor (32) may transmit the obtained retinal image to a diagnostic device. The processor (32) may transmit an image obtained through the imaging module (31) to a server device through the communication module (33) and obtain diagnostic information generated based thereon.

Although not shown, the client device may further include an output module. The output module may include a display that outputs video or images or a speaker that outputs audio. The output module may output video or image data obtained by the obtained imaging unit. The output module may output diagnostic information obtained from a diagnostic device.

Although not shown, the client device may further include an input module. The input module may obtain user input. For example, the input module may obtain user input requesting diagnostic information. The input module may obtain user information for evaluating diagnostic information obtained from a diagnostic device. In addition, the input module may obtain physical information of the subject (at least one of height, weight, age, sex, race, smoking status, blood pressure (for example, blood pressure level, presence or absence of hypertension), presence or absence of diabetes (or blood sugar level), cholesterol level).

In addition, although not shown, the client device may further include a storage module. The storage module may store images obtained by the imaging unit.

### 1.1.3. Overview of Diagnostic Process

A diagnostic process may be performed by the diagnostic system or diagnostic device disclosed in the present specification. The diagnostic process can be largely divided into a learning process for learning a diagnostic model used for diagnosis and a diagnostic process using the diagnostic model.

FIG. 6 is a diagram for explaining a diagnostic process according to one embodiment. Referring to FIG. 6, the diagnostic process according to one embodiment may include a learning process of obtaining and processing data (S11), learning a diagnostic model (S12), and obtaining parameters of the learned diagnostic model (S13), and a diagnostic process of obtaining diagnostic target data (S21) and obtaining diagnostic information (S23) using the diagnostic model (S22) learned based on the diagnostic target data.

More specifically, the learning process may include a data processing process of processing input learning image data to process it into a state that can be used for training the model, and a learning process of training the model using the processed data. The learning process may be performed by the learning device described above.

The diagnostic process may include a data processing process of processing input examination target image data to process it into a state capable of performing diagnosis using a diagnostic model, and a diagnostic process of performing diagnosis using the processed data. The diagnostic process may be performed by the diagnostic device or server device described above.

Hereinafter, each process will be described.

### 1.2. Learning Process

According to one embodiment, a process for training a diagnostic model may be provided. As a specific example, a process for training a diagnostic model that performs or assists diagnosis based on retinal images may be disclosed.

The learning process described below may be performed by the learning device described above.

### 1.2.1. Learning Unit

According to one embodiment, the learning process may be performed by a learning unit. The learning unit may be provided in the learning device described above.

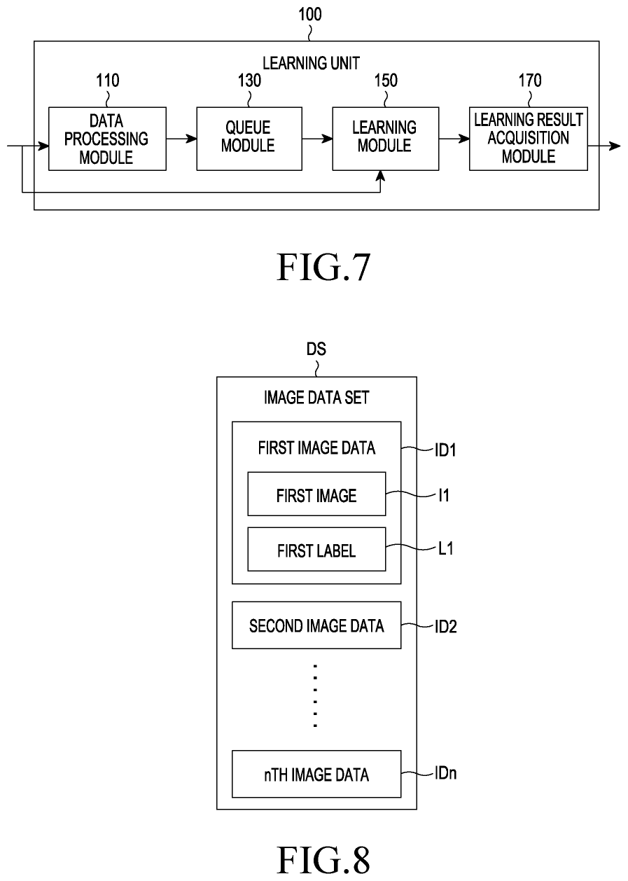

FIG. 7 is a diagram for explaining the configuration of a learning unit according to one embodiment. Referring to FIG. 7, the learning unit (100) may include a data processing module (110), a queue module (130), a learning module (150), and a learning result acquisition module (170). Each module may perform individual steps of the data processing process and the learning process as described later. However, the components described in FIG. 7 and the functions performed by each component are not all essential, and depending on the learning form, some components may be added or some components may be omitted.

### 1.2.2. Data Processing Process

### 1.2.2.1. Image Data Acquisition

According to one embodiment, a data set may be obtained. According to one embodiment, a data processing module may obtain a data set.

The data set may be an image data set.

As an example, it may be a retinal image data set. The retinal image data set may be obtained using a general non-mydriatic retinal camera, etc. The retinal image may be a panoramic retinal image or a wide retinal image. The retinal image may be a red-free image. The retinal image may be an infrared-photographed image. The retinal image may be an autofluorescence-photographed image. The image data may be obtained in any one of JPG, PNG, DCM (DICOM), BMP, GIF, and TIFF formats.

As another example, the data set may be an image data set including any one of an Optical Coherence Tomography (OCT) image, an OCT angiography image, or a retinal angiography image. In this case, a diagnostic model learned using a data set including an OCT image, an OCT angiography image, or a retinal angiography image may predict or output diagnostic information (or label) based on a target OCT image, a target OCT angiography image, or a target retinal angiography image.

The data set may include a training data set. The data set may include a test data set. The data set may include a validation data set. In other words, the data set may be allocated to at least one data set among a training data set, a test data set, and a validation data set.

The data set may be determined in consideration of diagnostic information to be obtained using a diagnostic model learned through the corresponding data set. For example, when it is desired to train a diagnostic model for obtaining diagnostic information related to cataracts, the obtained data set may be determined as an infrared retinal image data set. Alternatively, when it is desired to train a diagnostic model for obtaining diagnostic information related to macular degeneration, the obtained data set may be an autofluorescence-photographed retinal image data set.

Individual data included in the data set may include a label. There may be a plurality of labels. In other words, individual data included in the data set may be labeled for at least one feature. As an example, the data set is a retinal image data set including a plurality of retinal image data, and each retinal image data may include a diagnostic information label (for example, presence or absence of a specific disease) and/or finding information (for example, presence or absence of abnormality in a specific site) label according to the corresponding image.

As another example, the data set is a retinal image data set and each retinal image data may include a peripheral information label for the corresponding image. For example, each retinal image data may include a peripheral information label including left-right eye information regarding whether the corresponding retinal image is an image of the left eye or the right eye, sex information regarding whether it is a retinal image of a female or a male, age information regarding the age of the subject who photographed the corresponding retinal image, etc.

FIG. 8 is a conceptual diagram for explaining an image data set according to one embodiment. Referring to FIG. 8, the image data set (DS) according to one embodiment may include a plurality of image data (ID). Each image data (ID) may include an image (I) and a label (L) assigned to the image. Referring to FIG. 10, the image data set (DS) may include first image data (ID1) and second image data (ID2). The first image data (ID1) may include a first image (I1) and a first label (L1) corresponding to the first image.

In FIG. 8, the case where one image data includes one label is described as a reference, but as described above, one image data may include a plurality of labels.

### 1.2.2.2. Image Preprocessing

According to one embodiment, preprocessing of images may be performed. If images are used for learning as inputted, overfitting phenomena for unnecessary characteristics may occur and learning efficiency may also be degraded.

To prevent this, the data processing module may improve learning efficiency and performance by appropriately preprocessing image data to conform to the purpose of learning.

In one embodiment, the data processing module may perform preprocessing of images using or appropriately combining various techniques such as image resizing, grayscale conversion, histogram equalization, normalization, feature enhancement, image augmentation, noise removal, edge detection, segmentation, morphological operations, color space conversion, etc. Hereinafter, preprocessing of images using some techniques will be described.

### 1.2.2.2.1. Image Resizing

According to one embodiment, the size of obtained image data may be adjusted. That is, images may be resized. According to one embodiment, image resizing may be performed by the data processing module of the learning unit described above.

The size or aspect ratio of an image may be adjusted. A plurality of obtained images may have their sizes adjusted to have a constant size. Alternatively, images may have their sizes adjusted to have a constant aspect ratio. Resizing an image may be applying an image conversion filter to the image.

When the size or capacity of individual obtained images is excessively large or small, the size or capacity of the image may be adjusted to convert it to an appropriate size. Alternatively, when the sizes or capacities of individual images are various, the size or capacity may be unified through resizing.

According to one embodiment, the capacity of an image may be adjusted. For example, when the capacity of an image exceeds an appropriate range, the image may be reduced through down sampling. Alternatively, when the capacity of an image does not reach an appropriate range, the image may be enlarged through upsampling or interpolating.

According to another embodiment, the size or aspect ratio of an image may be adjusted by cropping the image or adding pixels to the obtained image. For example, when the image includes parts unnecessary for learning, a part of the image may be cropped to remove them. Alternatively, when a part of the image is cut off and the aspect ratio does not match, the image aspect ratio may be adjusted by adding columns or rows. In other words, the aspect ratio may be adjusted by adding margins or padding to the image.

According to yet another embodiment, the capacity and size or aspect ratio of an image may be adjusted together. As an example, when the capacity of an image is large, the image capacity may be reduced by down sampling the image, and unnecessary parts included in the reduced image may be cropped to convert it into appropriate image data.

In addition, according to another embodiment, the orientation of image data may also be changed.

As a specific example, when a retinal image data set is used as a data set, each retinal image may have its capacity adjusted or size adjusted. Cropping may be performed to remove margin parts excluding the retina part of the retinal image, or padding may be performed to adjust the aspect ratio by supplementing cut parts of the retinal image.

### 1.2.2.2.2. Feature Enhancement

According to one embodiment, the data processing module may perform preprocessing to emphasize features of retinal images. For example, the data processing module may perform preprocessing to facilitate detection of abnormal signs of ophthalmic disease in retinal images or preprocessing to emphasize retinal blood vessels or blood flow changes.

As an example, preprocessing of an image may be performed on an image for which the resizing processing described above has been completed. However, the content of the invention disclosed in the present specification is not limited thereto, and preprocessing on an image may be performed by omitting the resizing processing. Applying preprocessing to an image may be applying a preprocessing filter to the image.

According to one embodiment, a blur filter may be applied to an image. A Gaussian filter may be applied to an image. A Gaussian blur filter may be applied to an image. Alternatively, a deblur filter that sharpens an image may be applied to the image.

According to another embodiment, a filter that adjusts or modulates the color of an image may be applied. For example, a filter that changes the value of some components among RGB values constituting the image or that binarizes the image may be applied.

According to yet another embodiment, a filter that emphasizes a specific element in an image may be applied. For example, for retinal image data, preprocessing may be performed to emphasize vascular elements from each image. In this case, preprocessing to emphasize vascular elements may be applying one or more filters sequentially or in combination.

In addition, according to one embodiment, preprocessing of an image may be performed in consideration of characteristics of diagnostic information to be obtained. For example, when it is desired to obtain diagnostic information related to findings such as retinal hemorrhage, drusen, microaneurysm, exudate, etc., preprocessing for converting the obtained retinal image into a red-free retinal image form may be performed.

### 1.2.2.2.3. Image Augmentation

According to one embodiment, images may be augmented or expanded. Augmentation of images may be performed by the data processing module of the learning unit described above.

Augmented images may be used to improve training performance of a diagnostic model. For example, when the amount of data for training a diagnostic model is insufficient, the number of training image data may be increased by modulating existing training image data to expand the number of data for training and using the modulated (or changed) images together with original images. Accordingly, overfitting may be suppressed, the layers of the model may be formed deeper, and prediction accuracy may be improved.

For example, expansion of image data may be performed by horizontally flipping an image, cropping a part of an image, correcting color values of an image, or adding artificial noise. As a specific example, cropping a part of an image may be performed by cropping a partial region of elements constituting the image or randomly cropping partial regions. As more examples, image data may be expanded by horizontally flipping, vertically flipping, resizing by a certain ratio, cropping, padding, color adjusting, or brightness adjusting the image data.

In addition, in one embodiment, the data processing module may augment images by rotating retinal images. As the shape of the retina is circular, there may be no data loss even if the retina is rotated. Accordingly, when performing augmentation of images by rotating retinal images, a plurality of retinal images may be obtained without data loss. As an example, the data processing module may obtain a plurality of retinal images by rotating retinal images based on a predetermined angle (for example, 15 degrees, 30 degrees, 60 degrees, etc.).

In addition, in one embodiment, the augmentation or expansion of image data described above may generally be applied to a training data set. However, it may also be applied to other data sets, for example, a test data set, that is, a data set for testing a model after learning using training data and verification using verification data have been completed.

As a specific example, when a retinal image data set is used as a data set, an augmented retinal image data set may be obtained by randomly applying one or more processes among flipping, cropping, adding noise, or changing color of images to increase the number of data.

### 1.2.2.3. Image Serialization

According to one embodiment, image data may be serialized (linearization). Images may be serialized by the data processing module of the learning unit described above. The serialization module may serialize preprocessed image data and transfer it to a queue module.

When image data is used as it is for learning, decoding is required because the image data has an image file form such as JPG, PNB, DCM, etc., and if learning is performed through decoding each time, the performance of model learning may be degraded. Accordingly, learning may be performed by serializing the image file without using it as it is for learning. Therefore, serialization of image data may be performed to improve learning performance and speed. The image data to be serialized may be image data to which one or more steps of the image resizing and image preprocessing described above have been applied, or may be image data to which neither has been processed.

Each image data included in the image data set may be converted into string form. Image data may be converted into binarized data form. In particular, image data may be converted into a data form suitable for being used in learning a diagnostic model. As an example, image data may be converted into TFRecord form for use in learning a diagnostic model using tensorflow.

As a specific example, when a retinal image set is used as a data set, the obtained retinal image set may be converted into TFRecord form and used for learning a diagnostic model.

### 1.2.2.4. Queue

A queue may be used to resolve data bottlenecks. The queue module of the learning unit described above may store image data in a queue and transfer it to a learning model module.

Particularly, when a learning process is performed using a Central Processing Unit (CPU) and a Graphic Processing Unit (GPU) together, by using a queue, bottlenecks between the CPU and GPU may be minimized, access to the database may be smoothed, and memory usage efficiency may be improved.

The queue may store data used for learning a diagnostic model. The queue may store image data. The image data stored in the queue may be image data to which at least one of the data processing processes (that is, resizing, preprocessing, and augmentation) described above has been processed, or may be an image in an obtained state as it is.

The queue may store image data, preferably serialized image data as described above. The queue may store image data and supply image data to a diagnostic model. The queue may transfer image data to a diagnostic model in batch size units.

The queue may provide image data. The queue may provide data to a learning module to be described later. As data is extracted from the learning module, the number of data accumulated in the queue may decrease.

As learning of the diagnostic model progresses, when the number of data stored in the queue decreases below a reference, the queue may request replenishment of data. The queue may request replenishment of a specific type of data. The queue may replenish data in the queue when replenishment of data is requested by the learning unit.

The queue may be provided in system memory of the learning device. For example, the queue may be formed in Random Access Memory (RAM) of a central processing unit (CPU). In this case, the size, that is, capacity, of the queue may be determined according to the RAM capacity of the CPU. As the queue, a First In First Out (FIFO) queue, a Primary Queue, or a random queue may be used.

### 1.2.3. Learning Process

According to one embodiment, a learning process of a diagnostic model may be disclosed.

According to one embodiment, learning of a diagnostic model may be performed by the learning device described above. The learning process may be performed by the processor of the learning device described above. The learning process may be performed by the learning module of the learning unit described above.

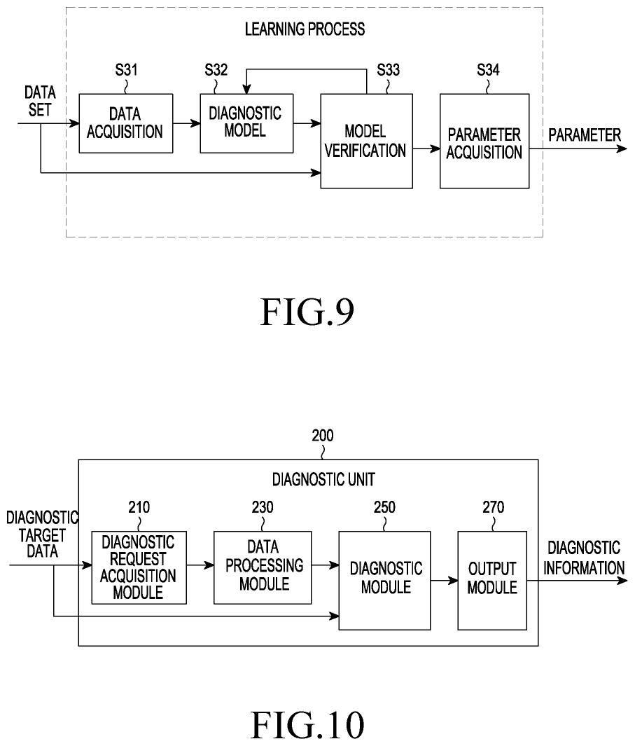

FIG. 9 is a block diagram for explaining a learning process of a diagnostic model according to one embodiment. Referring to FIG. 9, the learning process of a diagnostic model according to one embodiment may be performed by obtaining data (S31), learning a diagnostic model (S32), verifying the learned model (S33), and obtaining variables of the learned model (S34).

### 1.2.3.1. Data Input

A data set for learning a diagnostic model may be obtained.

The obtained data may be an image data set processed by the data processing process described above. As an example, the data set may include retinal image data that has been size-adjusted, to which preprocessing filters have been applied, and which has been serialized after data augmentation.

In the learning stage of the diagnostic model, a training data set may be obtained and used. In the verification stage of the diagnostic model, a verification data set may be obtained and used. In the test stage of the diagnostic model, a test data set may be obtained and used. Each data set may include retinal images and labels.

The data set may be obtained from a queue. The data set may be obtained from the queue in batch size units. For example, when 60 is designated as the batch size, the data set may be extracted from the queue in units of 60. The size of the batch size may be limited by the RAM capacity of the GPU.

The data set may be randomly obtained from the queue to the learning module. The data set may be obtained in the order accumulated in the queue.

The learning module may extract by specifying the configuration of the data set obtained from the queue. As an example, the learning module may extract so that retinal image data having a left eye label and retinal image data having a right eye label of a specific subject are used together for learning.

The learning module may obtain a data set of a specific label from the queue. As an example, the learning module may obtain a retinal image data set having an abnormal diagnostic information label from the queue. The learning module may obtain a data set by specifying the ratio of the number of data according to labels from the queue. As an example, the learning module may obtain a retinal image data set from the queue such that the number of retinal image data having an abnormal diagnostic information label and the number of retinal image data having a normal diagnostic information label is 1 to 1.

### 1.2.3.2. Model Design

The diagnostic model may be designed as various models such as a neural network model, machine learning model, etc. In one embodiment, when the diagnostic model includes a neural network model, the network model may include a plurality of layers.

The neural network model may be implemented in the form of a classifier that generates diagnostic information. The classifier may perform binary classification or multi-classification. For example, the neural network model may be a binary classification model that classifies input data into normal or abnormal classes for target diagnostic information such as a specific disease or abnormal sign. Alternatively, the neural network model may be a multi-classification model that classifies input data into a plurality of grade classes for a specific characteristic (for example, the degree of disease progression). Alternatively, the neural network model may be implemented as a regression type model that outputs a specific numerical value related to a specific disease.

The neural network model may include a Convolutional Neural Network (CNN). As a CNN structure, at least one of AlexNet, LENET, NIN, VGGNet, ResNet, WideResnet, GoogleNet, FractaNet, DenseNet, FitNet, RitResNet, HighwayNet, MobileNet, and DeeplySupervisedNet may be used. The neural network model may be implemented using a plurality of CNN structures.

As an example, the neural network model may be implemented to include a plurality of VGGNet blocks. As a more specific example, the neural network model may be provided by combining a first structure in which a CNN layer having 64 filters of 3×3 size, a Batch Normalization (BN) layer, and a ReLu layer are sequentially combined, and a second block in which a CNN layer having 128 filters of 3×3 size, a ReLu layer, and a BN layer are sequentially combined.

The neural network model may include a max pooling layer following each CNN block, and may include a Global Average pooling (GAP) layer, a Fully Connected (FC) layer, and an activation layer (for example, sigmoid, softmax, etc.) at the end.

In addition, in another embodiment, when the diagnostic model includes a machine learning model, the machine learning model may include a linear regression model, a Cox proportional hazards model, etc.

### 1.2.3.3. Model Learning

The diagnostic model may be learned using a training data set.

The diagnostic model may be learned using a labeled data set. However, the learning process of the diagnostic model described in the present specification is not limited thereto, and the diagnostic model may be learned in an unsupervised form using unlabeled data.

Learning of the diagnostic model may be performed by obtaining a result value using a diagnostic model to which arbitrary weight values are assigned based on training image data, comparing the obtained result value with a label value of the training data, and performing backpropagation according to the error to optimize the weight values. In addition, learning of the diagnostic model may be influenced by a verification result of the model to be described later, a test result, and/or feedback from a diagnostic stage.

The learning of the diagnostic model described above may be performed using TensorFlow. However, the present application is not limited thereto, and frameworks such as Theano, Keras, Caffe, Torch, and Microsoft Cognitive Toolkit (CNTK) may be used for learning the diagnostic model.

### 1.2.3.4. Model Validation

The diagnostic model may be verified using a verification data set. Verification of the diagnostic model may be performed by obtaining a result value for the verification data set from the diagnostic model on which learning has been performed, and comparing the result value with a label of the verification data set. Verification may be performed by measuring accuracy of the result value. According to the verification result, parameters (for example, weights and/or biases) or hyper parameters (for example, learning rate) of the diagnostic model may be adjusted.

As an example, the learning device according to one embodiment may train a diagnostic model that predicts diagnostic information based on retinal images, and perform verification of the diagnostic model by comparing diagnostic information for a verification retinal image of the learned model with a verification label corresponding to the verification retinal image.

For verification of the diagnostic model, a separate verification set (external data set), that is, a data set having distinguishing factors not included in the training data set, may be used. For example, the separate verification set may be a data set that is distinguished from the training data set by factors such as race, environment, age, sex, etc.

### 1.2.3.5. Model Testing

The diagnostic model may be tested using a test data set.

According to the learning process according to one embodiment, the diagnostic model may be tested using a test data set that is distinguished from the training data set and the verification data set. According to the test result, parameters (for example, weights and/or biases) or hyper parameters (for example, learning rate) of the diagnostic model may be adjusted.

As an example, the learning device according to one embodiment may obtain a result value using test retinal image data not used for training and verification as input from a diagnostic model learned to predict diagnostic information based on retinal images, and perform testing of the learned and verified diagnostic model.

For testing the diagnostic model, a separately provided verification set (external data set), that is, a data set having factors distinguished from training and/or verification data, may be used.

### 1.2.3.6. Output of Results

As a result of learning the diagnostic model, optimized parameter values of the model may be obtained. As described above, as learning of the model is repeatedly performed using the test data set, more appropriate parameter (or variable) values may be obtained. When learning has progressed sufficiently, optimized values of weights and/or biases may be obtained.

According to one embodiment, the learned diagnostic model and/or parameters or variables of the learned diagnostic model may be stored in the learning device and/or the diagnostic device (or server). The learned diagnostic model may be used for prediction of diagnostic information by the diagnostic device and/or the client device. In addition, parameters or variables of the learned diagnostic model may be updated by feedback obtained from the diagnostic device or the client device.

### 1.2.3.7. Model Ensemble

According to one embodiment, in the process of learning one diagnostic model, a plurality of sub-models may be learned simultaneously. The plurality of sub-models may have different hierarchical structures.

In this case, the diagnostic model according to one embodiment may be implemented by combining a plurality of sub-diagnostic models. In other words, learning of the diagnostic model may be performed using an ensemble technique that combines a plurality of sub-diagnoses.

When a diagnostic model is constructed by forming an ensemble, prediction may be performed by synthesizing results predicted from various types of sub-diagnostic models, so that accuracy of result prediction may be further improved.

### 1.3. Diagnostic Process

According to one embodiment, a diagnostic process (or diagnostic process) for obtaining diagnostic information using a diagnostic model may be provided. As a specific example, by the diagnostic process, diagnostic information (for example, diagnostic information or finding information) may be predicted using a retinal image and through a learned diagnostic model.

The diagnostic process described below may be performed by a diagnostic device.

### 1.3.1. Diagnostic Unit

According to one embodiment, the diagnostic process may be performed by the processor of the diagnostic device described above. The processor may be provided in the diagnostic device described above.

FIG. 10 is a diagram for explaining the configuration of a diagnostic unit according to one embodiment. Referring to FIG. 10, the diagnostic unit (200) may include a diagnostic request acquisition module (210), a data processing module (230), a diagnostic module (250), and an output module (270).

Each module may perform individual steps of the data processing process and the diagnostic process as described later. However, the components described in FIG. 10 and the functions performed by each component are not all essential, and some components may be added or some components may be omitted depending on the aspect of diagnosis.

### 1.3.2. Data Acquisition and Diagnostic Request

The diagnostic device according to one embodiment may obtain diagnostic target data and obtain diagnostic information based thereon. The diagnostic target data may be image data. Data acquisition and acquisition of a diagnostic request may be performed by the diagnostic request acquisition module of the diagnostic unit described above.

As an example, the diagnostic target data (TD) may include a diagnostic target image (TI) and diagnostic target subject information (PI; patient information).

The diagnostic target image (TI) may be an image for obtaining diagnostic information regarding a diagnostic target subject. For example, the diagnostic target image may be a retinal image and/or a fundus image. The diagnostic target image (TI) may have any one format of JPG, PNG, DCM (DICOM), BMP, GIF, and TIFF.

The diagnostic subject information (PI) may be information for identifying a diagnostic target subject. Alternatively, the diagnostic subject information (PI) may be characteristic information of the diagnostic target subject or image. For example, the diagnostic subject information (PI) may include information such as the imaging date and time of the diagnostic target image, imaging equipment, identification number of the diagnostic target subject, ID, name, sex, age, weight, race, smoking status, blood pressure (presence or absence of hypertension), presence or absence of diabetes, etc. When the diagnostic target image is a retinal image, the diagnostic subject information (PI) may further include ocular-related information such as binocular information indicating whether it is the left eye or the right eye.

The diagnostic device may obtain a diagnostic request. The diagnostic device may obtain diagnostic target data together with the diagnostic request. When a diagnostic request is obtained, the diagnostic device may obtain diagnostic information using a learned diagnostic model. The diagnostic device may obtain a diagnostic request from a client device. Alternatively, the diagnostic device may obtain a diagnostic request from a user through a separately provided input means.

### 1.3.3. Data Processing Process

The obtained data may be processed. Data processing may be performed by the data processing module of the diagnostic unit described above.

The data processing process may generally be performed similarly to the data processing process in the learning process described above. Hereinafter, the data processing process in the diagnostic process will be described focusing on differences from the data processing process in the learning process.

In the diagnostic process, the diagnostic device may obtain data as in the learning process. In this case, the obtained data may be in the same format as the data obtained in the learning process. For example, when the learning device trains a diagnostic model using image data in DCM format in the learning process, the diagnostic device may obtain a DCM image and obtain diagnostic information using the learned diagnostic model.

In the diagnostic process, the obtained diagnostic target image may be resized similarly to the image data used in the learning process. The diagnostic target image may have its form adjusted to have an appropriate capacity, size, and/or aspect ratio in order to efficiently perform prediction of diagnostic information through a learned diagnostic model.

For example, when the diagnostic target image is a retinal image, resizing such as cropping unnecessary parts of the image or reducing its size may be performed for prediction of diagnostic information based on the retinal image.

In the diagnostic process, a preprocessing filter may be applied to the obtained diagnostic target image, similarly to the image data used in the learning process. An appropriate filter may be applied to the diagnostic target image so that accuracy of diagnostic information prediction through a learned diagnostic model is further improved.

For example, when the diagnostic target image is a retinal image, preprocessing that facilitates prediction of diagnostic information, such as image preprocessing that emphasizes blood vessels or image preprocessing that emphasizes or weakens specific colors, may be applied to the diagnostic target image.

In the diagnostic process, the obtained diagnostic target image may be serialized similarly to the image data used in the learning process. The diagnostic target image may be converted or serialized into a form that facilitates driving the diagnostic model in a specific work frame.

Serialization of the diagnostic target image may be omitted. This may be because, unlike in the learning stage, in the diagnostic stage, the number of data processed by the processor at one time is not large, so the burden on data processing speed is relatively small.

In the diagnostic process, the obtained diagnostic target image may be stored in a queue similarly to the image data used in the learning process. However, since the number of processing data in the diagnostic process is smaller than in the learning process, the step of storing data in a queue may be omitted.

Meanwhile, in the diagnostic process, since an increase in the number of data is not required, data augmentation or image augmentation procedures may not be used, unlike in the learning process, for obtaining accurate diagnostic information.

### 1.3.4. Diagnostic Process

According to one embodiment, a diagnostic process using a learned diagnostic model may be disclosed. The diagnostic process may be performed in the diagnostic device described above. The diagnostic process may be performed in the diagnostic server described above. The diagnostic process may be performed in the control unit of the diagnostic device described above. The diagnostic process may be performed by the diagnostic module of the diagnostic unit described above.

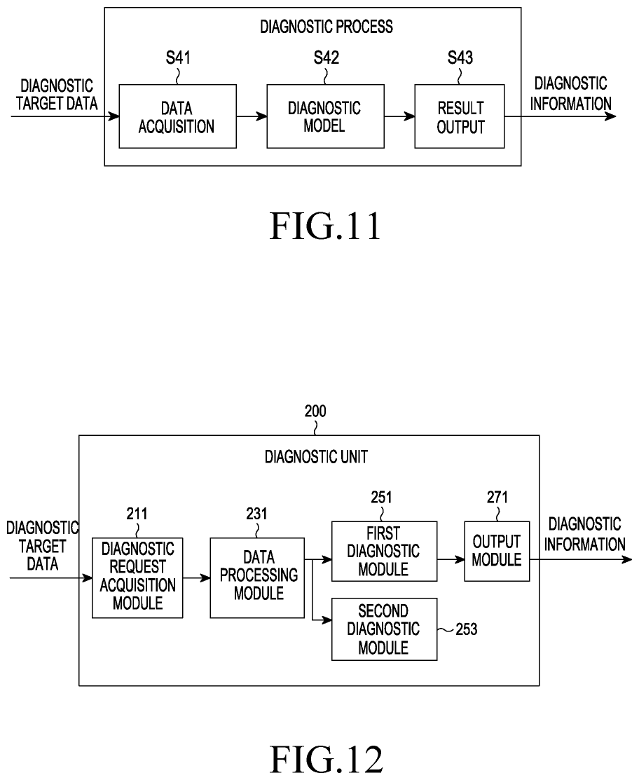

FIG. 11 is a diagram for explaining a diagnostic process according to one embodiment. Referring to FIG. 11, the diagnostic process may be performed by obtaining diagnostic target data (S31), using a learned diagnostic model (S42), and obtaining a result corresponding to the obtained diagnostic target data (S43). However, data processing may be selectively performed.

Hereinafter, referring to FIG. 11, each step of the diagnostic process will be described.

### 1.3.4.1. Data Input

According to one embodiment, the diagnostic module may obtain diagnostic target data. The obtained data may be processed data as described above. As an example, the obtained data may be retinal image data of a subject to which preprocessing has been applied to adjust size and emphasize blood vessels. According to one embodiment, a left eye image and a right eye image of one subject may be input together as diagnostic target data.

### 1.3.4.2. Data Classification

A diagnostic model provided in classifier form may classify an input diagnostic target image into a positive or negative class for a predetermined label.

The learned diagnostic model may receive diagnostic target data as input and output a predicted label. The learned diagnostic model may output a predicted value of diagnostic information. Diagnostic information may be obtained using the learned diagnostic model. Diagnostic information may be determined based on a predicted label.

For example, the diagnostic model may predict diagnostic information (that is, information regarding the presence or absence of disease) or finding information (that is, information regarding the presence or absence of abnormal findings) regarding an ophthalmic disease or systemic disease of a subject. In this case, diagnostic information or finding information may be output in probability form. For example, the probability that a subject has a specific disease or the probability that a retinal image of a subject has specific abnormal findings may be output. When using a diagnostic model provided in classifier form, a predicted label may be determined by considering whether an output probability value (or prediction score) exceeds a threshold value.

As a specific example, the diagnostic model may output the presence or absence of diabetic retinopathy of a subject as a probability value using the retinal image of the subject as a diagnostic target image. When using a diagnostic model in classifier form with 1 as normal, a retinal photograph of a subject may be input to the diagnostic model, and regarding whether the subject has diabetic retinopathy, a probability value of normal: abnormal may be obtained in a form such as 0.74:0.26.

Here, the case of classifying data using a diagnostic model in classifier form has been described as a reference, but the present invention is not limited thereto, and a specific diagnostic value (for example, blood pressure, etc.) may be predicted using a diagnostic model implemented in regression model form.

According to another embodiment, suitability information of an image may be obtained. The suitability information may indicate whether a diagnostic target image is suitable for obtaining diagnostic information using a diagnostic model.

The suitability information of an image may be quality information of an image. Quality information or suitability information may indicate whether a diagnostic target image reaches a reference level.

For example, when a diagnostic target image has defects due to defects in photographing equipment or the influence of lighting during photographing, an unsuitable result may be output as suitability information for the corresponding diagnostic target image. When a diagnostic target image includes noise above a certain level, the diagnostic target image may be determined to be unsuitable.

The suitability information may be a value predicted using a diagnostic model. Alternatively, the suitability information may be information obtained through a separate image analysis process.

According to one embodiment, even when an image is classified as unsuitable, diagnostic information obtained based on the unsuitable image may be obtained.

According to one embodiment, an image classified as unsuitable may be re-examined by a diagnostic model.

In this case, the diagnostic model performing re-examination may be different from the diagnostic model performing initial examination. For example, the diagnostic device may store a first diagnostic model and a second diagnostic model, and an image classified as unsuitable through the first diagnostic model may be examined through the second diagnostic model.

According to yet another embodiment, a map may be obtained from a learned diagnostic model. Diagnostic information may include a map. A map may be obtained together with other diagnostic information. For example, a map may include a Saliency Map, a Class Activation Map (CAM), a Heat Map, etc. In addition, in the case of CAM, it may be selectively obtained. For example, in the case of CAM, CAM may be extracted and/or output when diagnostic information or finding information obtained by a diagnostic model is classified into an abnormal class.

### 1.3.5. Output of Diagnostic Information

Diagnostic information may be determined based on results output from a diagnostic model. Output of diagnostic information may be performed by the output module of the diagnostic unit described above. Diagnostic information may be output from a diagnostic device to a client device. Diagnostic information may be output from a diagnostic device to a server device. Diagnostic information may be stored in a diagnostic device or diagnostic server. Diagnostic information may be stored in a separately provided server device, etc.

Diagnostic information may be managed in database form. For example, obtained diagnostic information may be stored and managed together with a diagnostic target image of the corresponding subject according to an identification number of the subject. In this case, the diagnostic target image and diagnostic information of the subject may be managed in chronological order. By managing diagnostic information and diagnostic target images in time series, tracking and history management of diagnostic information for each individual may be facilitated.

Diagnostic information may be provided to a user. Diagnostic information may be provided to a user through output means of a diagnostic device or client device. Diagnostic information may be output so that a user can recognize it through visual or auditory output means provided in a diagnostic device or client device.

According to one embodiment, an interface for effectively providing diagnostic information to a user may be provided.

In addition, when an image is classified as unsuitable, suitability information of the image may be provided together. As an example, when an image is classified as unsuitable, diagnostic information obtained according to the corresponding image and unsuitability determination information may be provided together.

A diagnostic target image determined to be unsuitable may be classified as a rephotographing target image. In this case, rephotographing guidance for the target subject of the image classified as a rephotographing target may be provided together with suitability information. Meanwhile, in response to providing diagnostic information obtained through a diagnostic model, feedback related to learning the diagnostic model may be obtained. For example, feedback for adjusting parameters or hyper parameters related to learning the diagnostic model may be obtained. Feedback may be obtained through an input module provided in a diagnostic device or client device.

According to one embodiment, diagnostic information corresponding to a diagnostic target image may include grade information. Grade information may be selected from a plurality of grades. Grade information may be determined based on diagnostic information and/or finding information obtained through a diagnostic model. Grade information may be determined in consideration of suitability information or quality information of a diagnostic target image. When the diagnostic model is a classifier model that performs multi-classification, grade information may be determined in consideration of the class into which the diagnostic target image is classified by the diagnostic model. When the diagnostic model is a regression model that outputs a numerical value related to a specific disease, grade information may be determined in consideration of the output numerical value. In addition, grade information may be determined by applying a predetermined cut-off value to a score according to diagnostic information.

For example, diagnostic information obtained corresponding to a diagnostic target image may include any one grade information selected from first grade information or second grade information. Grade information may be selected as first grade information when abnormal finding information or abnormal diagnostic information is obtained through a diagnostic model. Grade information may be selected as second grade information when abnormal finding information or abnormal diagnostic information is not obtained through a diagnostic model. Alternatively, grade information may be selected as first grade information when a numerical value obtained through a diagnostic model exceeds a reference numerical value, and may be selected as second grade information when the obtained numerical value does not reach the reference numerical value. First grade information may indicate that stronger abnormal information exists in the diagnostic target image compared to second grade information.

Meanwhile, grade information may be selected as third grade information when it is determined using image analysis or a diagnostic model that the quality of the diagnostic target image is below a reference. Alternatively, diagnostic information may include third grade information together with first or second grade information.

### 1.4. Diagnostic System Using Multiple Diagnostic Models

According to one embodiment, diagnostic information may be output using a diagnostic model as described above. The diagnostic model described above may be composed of one diagnostic model or may be composed of a plurality of diagnostic models. And, when the diagnostic model is composed of a plurality of diagnostic models, the plurality of diagnostic models may be configured in parallel or may be configured in series. Hereinafter, parallel diagnostic models and serial diagnostic models will be described.

### 1.4.1.1. Parallel Diagnostic System Configuration

According to one embodiment, a parallel diagnostic system for obtaining a plurality of diagnostic information may be provided. The parallel diagnostic system may train a plurality of diagnostic models for obtaining a plurality of diagnostic information, and obtain a plurality of diagnostic information using the plurality of learned diagnostic models.

For example, the parallel diagnostic system may train a first diagnostic model that obtains first diagnostic information related to the presence or absence of ophthalmic disease of a subject and a second diagnostic model that obtains second diagnostic information related to the presence or absence of systemic disease of a subject based on retinal images, and output diagnostic information regarding the presence or absence of ophthalmic disease and the presence or absence of systemic disease of a subject using the learned first diagnostic model and second diagnostic model.

The plurality of diagnostic models may be learned in parallel and/or independently. By learning models to predict different labels through a plurality of diagnostic models in this way, prediction accuracy for each label may be improved and efficiency of prediction operations may be increased. Since matters previously described may be applied to learning of a plurality of diagnostic models, detailed description will be omitted.

### 1.4.1.2. Parallel Diagnostic Process

According to one embodiment, a diagnostic process for obtaining a plurality of diagnostic information may be provided. The diagnostic process for obtaining a plurality of diagnostic information may be implemented in the form of a parallel diagnostic process including a plurality of mutually independent diagnostic processes.

According to one embodiment, the diagnostic process may be performed by a plurality of diagnostic modules. Each diagnostic process may be performed independently.

FIG. 12 is a block diagram for explaining a diagnostic unit according to one embodiment.

Referring to FIG. 12, the diagnostic unit (200) according to one embodiment may include a diagnostic request acquisition module (211), a data processing module (231), a first diagnostic module (251), a second diagnostic module (253), and an output module (271). Each module of the diagnostic unit (200) may operate similarly to the diagnostic module of the diagnostic unit shown in FIG. 10 unless otherwise specifically mentioned.

In FIG. 12, even when the diagnostic unit (200) includes a plurality of diagnostic modules, the diagnostic request acquisition module (211), data processing module (231), and output module (271) are shown as common, but the present invention is not limited to such a configuration, and the diagnostic request acquisition module, data processing module, and/or output module may also be provided in plurality. The plurality of diagnostic request acquisition modules, data processing modules, and/or output modules may also operate in parallel.

For example, the diagnostic unit (200) may include a first data processing module that performs first processing on an input diagnostic target image and a second processing module that performs second data processing on the diagnostic target image, and the first diagnostic module may obtain first diagnostic information based on the first processed diagnostic target image, and the second diagnostic module may obtain second diagnostic information based on the second processed diagnostic target image. The first processing and/or second processing may be any one selected from image resizing, color modulation of image, blur filter application, blood vessel emphasis processing, red-free conversion, partial region cropping, and partial element extraction.

The plurality of diagnostic modules may obtain different diagnostic information. The plurality of diagnostic modules may obtain diagnostic information using different diagnostic models. For example, the first diagnostic module may obtain first diagnostic information related to whether a subject corresponds to ophthalmic disease using a first diagnostic model that predicts whether the subject corresponds to ophthalmic disease, and the second diagnostic module may obtain second diagnostic information related to whether the subject corresponds to systemic disease using a second diagnostic model that predicts whether the subject corresponds to systemic disease.

As a more specific example, the first diagnostic module may obtain first diagnostic information regarding whether a subject corresponds to diabetic retinopathy using a first diagnostic model that predicts whether the subject corresponds to diabetic retinopathy based on retinal images, and the second diagnostic module may obtain second diagnostic information related to whether the subject corresponds to hypertension using a second diagnostic model that predicts whether the subject corresponds to hypertension based on retinal images.

In addition, the diagnostic process according to one embodiment may include a plurality of sub-diagnostic processes. Each sub-diagnostic process may be performed using different diagnostic models. Each sub-diagnostic process may be performed in different diagnostic processes. For example, the first diagnostic module may perform a first sub-diagnostic process for obtaining first diagnostic information through a first diagnostic model. Alternatively, the second diagnostic module may perform a second sub-diagnostic process for obtaining second diagnostic information through a second diagnostic model.

The plurality of learned diagnostic models may receive diagnostic target data as input and output predicted labels or probabilities. Each diagnostic model may be provided in classifier form and may classify input diagnostic target data for a predetermined label. In this case, the plurality of diagnostic models may be provided in classifier form learned for different characteristics.

Meanwhile, a map may be obtained from each diagnostic model, and the map may be selectively obtained. The map may be extracted when predetermined conditions are satisfied. For example, when the first diagnostic information indicates that the subject is abnormal for the first characteristic, a first map may be obtained from the first diagnostic model.

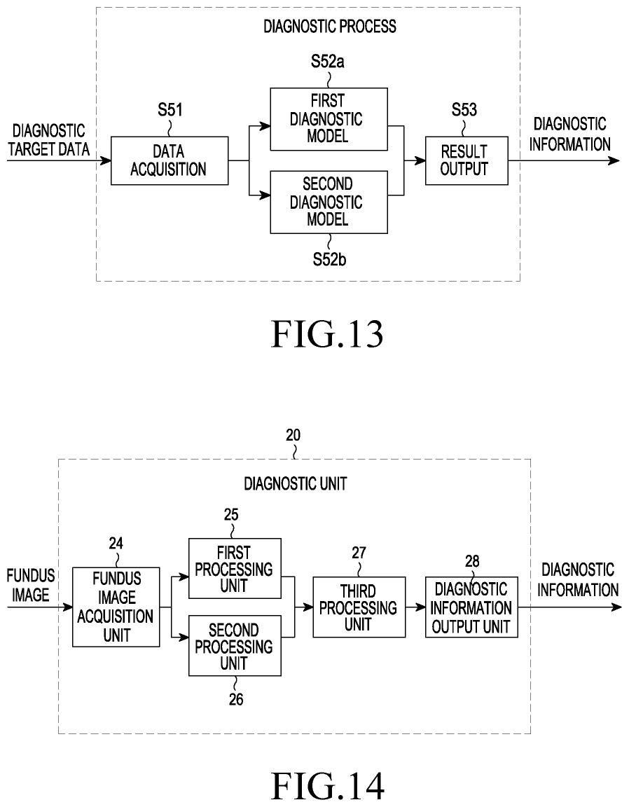

FIG. 13 is a diagram for explaining a diagnostic process according to one embodiment.

Referring to FIG. 13, the diagnostic process according to one embodiment may include obtaining diagnostic target data (S51) and obtaining diagnostic information according to the diagnostic target data using a first diagnostic model and a second diagnostic model (S51a, S51b) (S53). The diagnostic target data may be processed data.

The diagnostic process according to one embodiment may include obtaining first diagnostic information through a learned first diagnostic model and obtaining second diagnostic information through a learned second diagnostic model. The first diagnostic model and the second diagnostic model may obtain first diagnostic information and second diagnostic information, respectively, based on the same diagnostic target data.

For example, the first diagnostic model and the second diagnostic model may obtain first diagnostic information regarding ophthalmic disease of a subject and second diagnostic information regarding whether the subject has cardiovascular disease, respectively, based on a diagnostic target retinal image.

In addition, unless otherwise specifically mentioned, the diagnostic process described in relation to FIG. 13 may be implemented similarly to the diagnostic process described above in relation to FIG. 11.

### 1.4.1.3. Output of Diagnostic Information

According to one embodiment, diagnostic information obtained by a parallel diagnostic process may be obtained. The obtained diagnostic information may be stored in a diagnostic device, server device, and/or client device. The obtained diagnostic information may be transferred to an external device.

The plurality of diagnostic information may respectively indicate a plurality of labels predicted by a plurality of diagnostic models. The plurality of diagnostic information may correspond, respectively, to a plurality of labels predicted by a plurality of diagnostic models. Alternatively, diagnostic information may be information determined based on a plurality of labels predicted by a plurality of diagnostic models. Diagnostic information may correspond to a plurality of labels predicted by a plurality of diagnostic models.

In other words, the first diagnostic information may be diagnostic information corresponding to a first label predicted through a first diagnostic model. Alternatively, the first diagnostic information may be diagnostic information determined by considering together a first label predicted through a first diagnostic model and a second label predicted through a second diagnostic model.

Meanwhile, map images obtained from a plurality of diagnostic models may be output. A map image may be output when predetermined conditions are satisfied. For example, in any one case where the first diagnostic information indicates that the subject is abnormal for the first characteristic or the second diagnostic information indicates that the subject is abnormal for the second characteristic, a map image obtained from the diagnostic model from which diagnostic information indicated abnormality was output may be output.

A plurality of diagnostic information and/or map images may be provided to a user. The plurality of diagnostic information, etc. may be provided to a user through output means of a diagnostic device or client device.

According to one embodiment, diagnostic information corresponding to a diagnostic target image may include grade information. Grade information may be selected from a plurality of grades. Grade information may be determined based on a plurality of diagnostic information and/or finding information obtained through a diagnostic model. Grade information may be determined in consideration of suitability information or quality information of a diagnostic target image. Grade information may be determined in consideration of classes into which a diagnostic target image is classified by a plurality of diagnostic models. Grade information may be determined in consideration of numerical values output from a plurality of diagnostic models.

For example, diagnostic information obtained corresponding to a diagnostic target image may include any one grade information selected from first grade information or second grade information. Grade information may be selected as first grade information when, among diagnostic information obtained through a plurality of diagnostic models, at least one abnormal finding information or abnormal diagnostic information is obtained. Grade information may be selected as second grade information when, among diagnostic information obtained through diagnostic models, abnormal finding information or abnormal diagnostic information is not obtained.

Grade information may be selected as first grade information when at least one of numerical values obtained through a diagnostic model exceeds a reference numerical value, and may be selected as second grade information when all obtained numerical values do not reach the reference numerical value. First grade information may indicate that stronger abnormal information exists in the diagnostic target image compared to second grade information.

Grade information may be selected as third grade information when it is determined using image analysis or a diagnostic model that the quality of the diagnostic target image is below a reference. Alternatively, diagnostic information may include third grade information together with first or second grade information.

In addition, the diagnostic system according to one embodiment may include a retinal image acquisition unit, a first processing unit, a second processing unit, a third processing unit, and a diagnostic information output unit.

According to one embodiment, the diagnostic system may include a diagnostic device. The diagnostic device may include a retinal image acquisition unit, a first processing unit, a second processing unit, a third processing unit, and/or a diagnostic information output unit. However, the present invention is not limited thereto, and each unit included in the diagnostic system may be respectively located at appropriate positions on a learning device, diagnostic device, learning diagnostic server, and/or client device. Hereinafter, for convenience, description will be made based on the case where the diagnostic device of the diagnostic system includes a retinal image acquisition unit, a first processing unit, a second processing unit, a third processing unit, and a diagnostic information output unit.

FIG. 14 is a diagram for explaining a diagnostic system according to one embodiment. Referring to FIG. 14, the diagnostic system includes a diagnostic device, and the diagnostic device may include a retinal image acquisition unit, a first processing unit, a second processing unit, a third processing unit, and a diagnostic information output unit.

According to one embodiment, a diagnostic system for assisting diagnosis of a plurality of diseases based on retinal images may include: a retinal image acquisition unit that obtains a target retinal image that serves as a basis for obtaining diagnostic information regarding a subject; a first processing unit that obtains a first result for the subject using a first diagnostic model—the first diagnostic model is machine-learned based on a first retinal image set—for the target retinal image; a second processing unit that obtains a second result for the subject using a second diagnostic model—the second diagnostic model is machine-learned based on a second retinal image set that is at least partially different from the first retinal image set—for the target retinal image; a third processing unit that determines diagnostic information regarding the subject based on the first result and the second result; and a diagnostic information output unit that provides the determined diagnostic information to a user.

The third processing unit may determine whether diagnostic information according to the target retinal image is normal information or abnormal information by considering the first result and the second result together.

The third processing unit may determine diagnostic information regarding the subject by giving priority to abnormal results so that diagnostic accuracy is improved.

The third processing unit may determine diagnostic information as normal when the first result is normal and the second result is normal, and may determine diagnostic information as abnormal when the first result is not normal or the second result is not normal.

The first result and the second result may be for the same disease or may be for different diseases.

At least one map related to a first/second result may be obtained through the first processing unit and/or the first/second diagnostic model, and the diagnostic information output unit may output an image of at least one map.

The diagnostic information output unit may output an image of at least one map when the diagnostic information obtained by the third processing unit is abnormal diagnostic information.

The diagnostic system may further include a fourth processing unit that obtains quality information of the target retinal image, and the diagnostic information output unit may output quality information of the target retinal image obtained by the fourth processing unit.

When it is determined in the fourth processing unit that quality information of the target retinal image is below a predetermined quality level, the diagnostic information output unit may provide information indicating that quality information of the target retinal image is below the predetermined quality level together with the determined diagnostic information to the user.

### 1.4.2.1. Configuration of Serial Diagnostic System

According to one embodiment, a serial diagnostic system in a form in which a plurality of diagnostic models are connected in series may be provided. Hereinafter, some embodiments of serial type diagnostic models will be described.

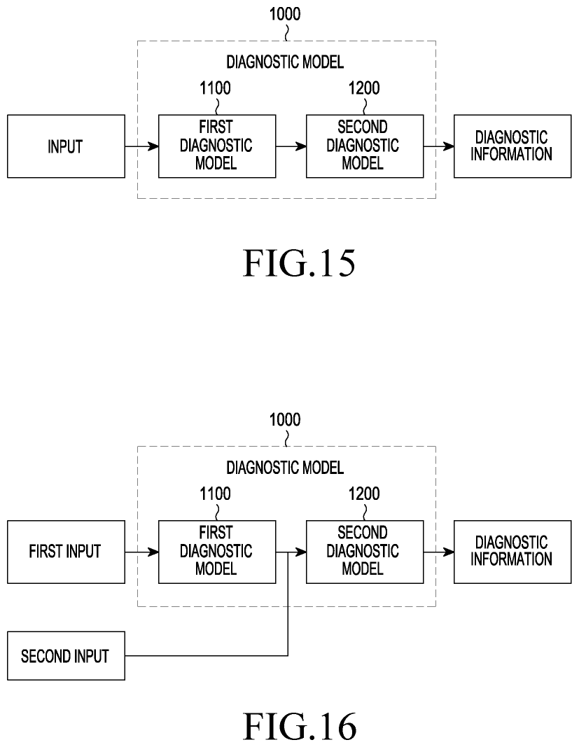

FIG. 15 is a diagram for explaining a serial diagnostic model according to one embodiment. Referring to FIG. 15, the diagnostic model (1000) may include a first serial model (1100) and a second serial model (1200).

The first diagnostic model (1100) may obtain input data including a retinal image and obtain a first output (or intermediate output).

The first diagnostic model (1100) may obtain input data including retinal images and/or other medical diagnostic images and/or non-visual diagnostic data. The input data may include retinal images, OCT images, iris images, ocular angiography images, lung CT images, lung CT images, heart CT images, lung X-ray images, heart X-ray images, kidney X-ray images, other tomography images, MRI images, or X-ray images. The input data may include data representing the age, height, sex, smoking status, family history, etc. of a subject.

The first output may be a value obtained by an output layer of the first diagnostic model (1100). For example, the first diagnostic model (1100) is a classifier model and the first output may include output values at a plurality of nodes of the output layer of the first diagnostic model (1100). Also, for example, the first diagnostic model (1100) is a regression model and the first output may include a numerical value obtained by the first diagnostic model (1100).

The first output may be a value provided by some layers of the first diagnostic model (1100). For example, the first output may be a value obtained based on a value of the output layer of the first diagnostic model (1100). Alternatively, the first output may be a value obtained based on a value of a hidden layer of the first diagnostic model (1100).

According to one embodiment, the first output may be a value obtained by an activation function in the output layer of the first diagnostic model (1100). When the output layer of the first diagnostic model (1100) includes a plurality of nodes (or neurons), the first output may include an output value according to each of the plurality of nodes or a value obtained through a predetermined function (for example, summation) based on each output value.

The activation function may be any one of a sigmoid function, hyperbolic tangent function, Rectified Linear Unit (ReLu) function, PReLu, Leaky ReLU function, Identity Function, Exponential Linear Unit (ELU) function, and Maxout function.

The first output may be a feature map or feature value related to a target disease. The first output may be a probability map, saliency map, heat map, etc. related to a target disease. The second diagnostic model (1200) may be provided to obtain diagnostic information based on a feature map or feature value related to a target disease.

The first output may be a probability phenotype related to a target disease. For example, when the target disease is coronary artery disease and diagnostic information is numerical information related to the target coronary artery disease, the first output may be the probability that the subject corresponds to target coronary artery disease obtained based on an ocular image. The second diagnostic model (1200) may be provided to obtain diagnostic information regarding the target disease based on probability expression related to the target disease.

The second diagnostic model (1200) may obtain a second output (or diagnostic information) based on the first output. The second diagnostic model (1200) may be a diagnostic model (1000) learned to obtain the second output using the first output as input. The second output may be diagnostic information in various forms described in the present specification.

FIG. 16 is a diagram for explaining a serial diagnostic model according to another embodiment. Referring to FIG. 16, the diagnostic model (1000) may include a first diagnostic model (1100) and a second diagnostic model (1200).

The first diagnostic model (1100) may obtain input data including an ocular image and obtain a first output (intermediate output). The second diagnostic model (1200) may obtain diagnostic information based on the first output and a second input.

The second input may be the same input data as the first input. For example, the first diagnostic model (1100) may obtain a first output based on an ocular image, and the second diagnostic model (1200) may obtain diagnostic information based on the first output and the ocular image.

The second input may be input data obtained based on the first input. For example, the second input may be input data obtained by performing image processing on an ocular image. For example, the second input may be a black-and-white processed ocular image, an ocular image with emphasized blood vessels, a blood vessel image extracted from an ocular image, or an ocular image with blood vessels removed.

The second input may be input data at least partially different from the first input.

The second input may be image data different from the first input. The second input may include retinal images, OCT images, iris images, ocular angiography images, lung CT images, lung CT images, heart CT images, lung X-ray images, heart X-ray images, kidney X-ray images, other tomography images, MRI images, or X-ray images.

The second input may include non-visual information regarding a subject. For example, the first diagnostic model (1100) may obtain a first output regarding a target disease based on an ocular image, and the second diagnostic model (1200) may obtain diagnostic information based on the first output and a second input (for example, physical information of the subject (at least one of height, weight, age, sex, race, smoking status, blood pressure (for example, blood pressure level, presence or absence of hypertension), presence or absence of diabetes (or blood sugar level), cholesterol level).

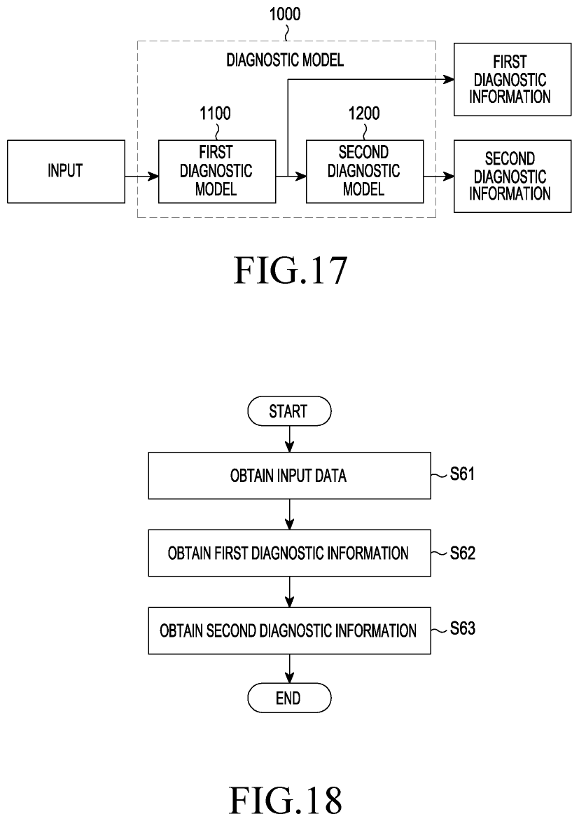

FIG. 17 is a diagram for explaining a serial diagnostic model according to yet another embodiment. Referring to FIG. 17, the diagnostic model (1000) may include a first diagnostic model (1100) and a second diagnostic model (1200).

Compared to FIG. 16, first diagnostic information obtained by the first diagnostic model (1100) may be further obtained by the diagnostic model (1000). The diagnostic model (1000) may obtain intermediate diagnostic information and secondary diagnostic information obtained based on the intermediate diagnostic information.

The diagnostic model (1000) may obtain first diagnostic information and second diagnostic information. The first diagnostic model (1100) may obtain input data including an ocular image and obtain a first output (first diagnostic information or intermediate output). The diagnostic model (1000) may obtain first diagnostic information based on the first output. The second diagnostic model (1200) may obtain second diagnostic information based at least in part on the first output. The diagnostic model (1000) may obtain second diagnostic information based at least in part on the first output and by considering together other information extracted from the ocular image.

The first diagnostic information and the second diagnostic information may be diagnostic information regarding the same target disease. The first diagnostic information may be diagnostic information that can be obtained (clinically or through a machine-learned model) based on ocular images, such as probability expressions for the presence or absence of ophthalmic disease, vascular abnormalities, presence or absence of cardiovascular disease, etc.

The second diagnostic information may represent more detailed diagnostic information than the first diagnostic information. For example, the second diagnostic information may include grade information representing the risk level for the target disease or score information representing a score related to the target disease, for the same target disease as the first diagnostic information.

The second diagnostic information may be associated with the first diagnostic information and may include diagnostic information that can be obtained by further considering information other than images, such as disease progression rate, guide information related to diagnostic information, etc.

Meanwhile, the first diagnostic information and the second diagnostic information may be diagnostic information regarding different diseases. The first diagnostic information and the second diagnostic information may be diagnostic information regarding different diseases belonging to the same group. For example, the first diagnostic information may be diagnostic information related to glaucoma belonging to the ophthalmic disease group and the second diagnostic information may be diagnostic information related to macular degeneration belonging to the ophthalmic disease group. For example, the first diagnostic information may be diagnostic information related to drusen belonging to the ophthalmic disease group and the second diagnostic information may be diagnostic information related to diabetic retinopathy belonging to the ophthalmic disease group.

The first diagnostic information and the second diagnostic information may be diagnostic information regarding different diseases belonging to different groups. For example, the first diagnostic information may be diagnostic information related to macular degeneration or drusen, etc. belonging to the ophthalmic disease group, and the second diagnostic information may be diagnostic information related to hyperlipidemia belonging to the cardiovascular disease group.

In the above embodiments, description has been made based on the case where the diagnostic model (1000) has a first diagnostic model (1100) and a second diagnostic model (1200), but the diagnostic model (1000) may include a greater number of diagnostic models. In addition, each diagnostic model may be connected through the parallel connection or serial connection described above.

### 1.4.2.2. Diagnosis Through Serial Diagnostic Model

According to one embodiment of the invention described in the present specification, a diagnostic method using a diagnostic model including serially connected sub-models may be provided.

FIG. 18 is a diagram for explaining a diagnostic method using a diagnostic model according to one embodiment.

Referring to FIG. 18, the diagnostic method according to one embodiment may include obtaining input data (S61), obtaining first diagnostic information (S62), and obtaining second diagnostic information (S63).

The step of obtaining input data (S61) may include obtaining a retinal image of a subject. The step of obtaining input data (S61) may further include obtaining a medical image for a body part other than the eye of the subject. The step of obtaining input data (S61) may further include obtaining non-visual information (for example, physical information of the subject) regarding the subject. The step of obtaining input data (S61) may further include performing preprocessing necessary for obtaining diagnostic information on an ocular image of the subject.

The step of obtaining first diagnostic information (S62) may include obtaining first diagnostic information regarding a subject based on an ocular image and through a first diagnostic model. Obtaining first diagnostic information may be obtaining diagnostic information related to a first disease. For example, obtaining first diagnostic information may include obtaining first diagnostic information representing the probability that a coronary artery calcium score of a subject is 0 or more based on an ocular image.

The step of obtaining second diagnostic information (S63) may include obtaining second diagnostic information regarding a subject based on the first diagnostic information and through a second diagnostic model.

Obtaining second diagnostic information may include obtaining diagnostic information that is related to a first disease and different from the first diagnostic information. For example, obtaining first diagnostic information may include obtaining first diagnostic information representing the probability that a coronary artery calcium score of a subject is 0 or more, and obtaining second diagnostic information may include obtaining second diagnostic information representing a score (for example, the probability of occurrence of cardiovascular disease-related events within 10 years) related to whether the subject has a target cardiovascular disease based on the first diagnostic information of the subject.

Alternatively, obtaining second diagnostic information may include obtaining diagnostic information related to a second disease different from the first disease. For example, obtaining first diagnostic information may include obtaining first diagnostic information representing whether a subject has a target ophthalmic disease, and obtaining second diagnostic information may include obtaining second diagnostic information representing whether the subject has a cardio-cerebrovascular disease.

Regarding diagnosis through the diagnostic model described above, it will be described in detail with specific examples below.

### 2. CARDIOVASCULAR DISEASE DIAGNOSIS METHOD

### 2.1.1. Biomarkers for Cardiovascular Disease

In one embodiment, cardiovascular disease biomarkers for predicting, measuring, and confirming the presence or absence and progression degree, etc. of cardiovascular disease may be used in a cardiovascular disease diagnosis method. In the present specification, cardiovascular disease biomarkers may include risk assessment tools for cardiovascular disease.

For example, the cardiovascular disease biomarkers may include Coronary Artery Calcium (CAC) score, Pooled Cohort Equation (PCE) score, QRISK score, modified Framingham Score (FRS), Carotid Intima-Media Thickness (CIMT) score, brachial-ankle pulse wave velocity (baPWV) score, etc.

In the case of the coronary artery calcium score (or cardiac calcification index), it may be used as a determination index for the calcification of the coronary artery. When the coronary artery becomes calcified as plaque accumulates in blood vessels, the walls of the cardiac blood vessels become narrower, which causes various heart diseases such as coronary artery disease, myocardial infarction, angina, and ischemic heart disease; thus, the coronary artery calcium index may be used as the basis for determining the risk of various heart diseases. For example, when the coronary artery calcium score value is large, it may be determined that the risk of coronary artery disease is high. In particular, the coronary artery calcium score is directly related to heart disease, particularly coronary artery disease (cardiac calcification), compared to factors indirectly related to heart disease such as smoking status, age, and sex, and may be used as a strong biomarker for heart health. Based on the coronary artery calcium score, the risk of cardiovascular disease may be classified as very low risk (coronary artery calcium score 0), somewhat increased risk (1 to 99), moderate risk (100 to 299), and high risk (300 or more).

The PCE score is an index for estimating the risk of onset of Atherosclerotic Cardiovascular Disease (ASCVD) within 10 years of an individual, and is used in guidelines as the basis for prescribing hypertension drugs such as statins. The PCE score may be used mainly as a cardiovascular disease biomarker in the United States. As an example, based on the PCE score, the risk of cardiovascular disease may be classified as low risk (PCE score 0% to 5%), borderline risk (5% to 7.4%), moderate risk (7.5% to 19.9%), and high risk (20% or more).

The QRISK score may be an index for estimating the risk of onset of cardiovascular disease within 10 years of an individual. QRISK is a cardiovascular disease biomarker mainly used in the United Kingdom and has several versions. In the present specification, QRISK 3 will be described for convenience of description. QRISK3 is an official risk assessment tool developed using data of 1.28 million individuals, and currently the UK guidelines recommend administration of stage 1 hypertension drugs when the QRISK3 score is 10%. As an example, using the QRISK3 score, the risk of cardiovascular disease may be classified as low risk (QRISK3 score 0 to 10%), moderate risk (10% to 20%), and high risk (20% or more).

The modified Framingham risk score may be an index for estimating the risk of onset of cardiovascular disease within 10 years of an individual. The modified Framingham risk score may be mainly used in Singapore. As an example, using the modified Framingham score, the risk of cardiovascular disease may be classified as low risk (QRISK3 score 0 to 10%), moderate risk (10% to 20%), and high risk (20% or more). These risk groups guide various treatment guidelines in Singapore, and are particularly used in prescribing recommended LDL cholesterol-related drugs.

The Carotid Intima-Media Thickness (CIMT) score is a measurement of the thickness between the inner and middle layers of the carotid artery by ultrasound, and may be used as a cardiovascular disease biomarker. As an example, using the CIMT score, the risk of cardiovascular disease may be classified as low risk (carotid intima-media thickness score less than 0.5 mm), borderline risk (0.5 mm to 0.75 mm), and high risk (0.75 mm or more).

The brachial-ankle pulse wave velocity (baPWV) score relates to the speed at which the pulse wave travels between the brachium and the ankle, and may be used as a biomarker for cardiovascular disease. As an example, when the baPWV score is 1,400 cm/s or more, the risk of cardiovascular disease may be estimated to be high.

In addition, various cardiovascular disease biomarkers may be used in the cardiovascular disease diagnosis method.

### 2.1.2. Cardiovascular Disease Diagnosis Method

The cardiovascular disease diagnosis method according to one embodiment of the present specification may be performed using at least one of the one diagnostic model, the parallel diagnostic model, or the serial diagnostic model described above. Hereinafter, for convenience of description, the description will focus on performing the cardiovascular disease diagnosis method using the serial diagnostic model.

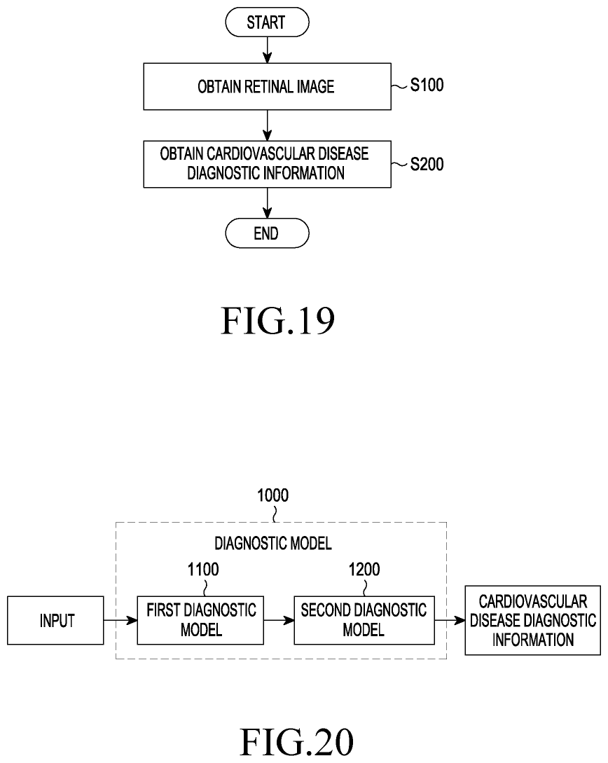

FIG. 19 is a diagram for explaining a cardiovascular disease diagnosis method according to one embodiment.

Referring to FIG. 19, the processor of the diagnostic device may include a step of obtaining a retinal image (S100) and a step of obtaining cardiovascular disease diagnostic information (S200).

In step S100, the processor of the diagnostic device may obtain a retinal image. In addition, according to embodiments, the processor of the diagnostic device may perform preprocessing, augmentation, serialization, etc. on the obtained retinal image. Since the content described above may be applied thereto, detailed description will be omitted.

In addition, in step S200, the processor of the diagnostic device may obtain cardiovascular disease diagnostic information. In the present specification, the cardiovascular disease diagnostic information may be expressed as Reti-CVD. The cardiovascular disease diagnosis method will be described using FIG. 20.

FIG. 20 illustrates a diagnostic model for obtaining cardiovascular disease diagnostic information according to one embodiment.

Referring to FIG. 20, the diagnostic model (1000) may be included in the processor and/or storage module of the diagnostic device. In addition, the description of the diagnostic model of FIGS. 15 to 18 may be applied to the diagnostic model (1000).

The diagnostic model (1000) may include a first diagnostic model (1100) and a second diagnostic model (1200). The first diagnostic model (1100) and the second diagnostic model (1200) may be machine learning models. In addition, the first diagnostic model (1100) and the second diagnostic model (1200) may be models based on the same algorithm or may be models based on different algorithms. For example, the first diagnostic model (1100) and the second diagnostic model (1200) may be neural network models. In addition, the first diagnostic model (1100) may be a neural network model, and the second diagnostic model (1200) may be a machine learning model that is not a neural network model. Hereinafter, for convenience of description, the description will focus on the case where the first diagnostic model (1100) is a neural network model and the second diagnostic model (1200) is a machine learning model that is not a neural network model, but the description of the present specification is not limited thereto.

The processor of the diagnostic device may input a retinal image (or a preprocessed retinal image, or a serialized retinal image) to the first diagnostic model (1100). Then, the processor of the diagnostic device may obtain a probability value and/or grade of the probability that the subject of the retinal image has a coronary artery calcium score of 0 or more from the first diagnostic model (1100). Here, the probability value may be a number between 0 and 1.

Specifically, the first diagnostic model (1100) may be a neural network model of CNN structure. In one embodiment, the first diagnostic model (1100) may use a 7×7 size kernel to consider more regions in judgment than a general CNN-structured neural network model. In addition, the first diagnostic model (1100) may calculate probability values for three cutoff values (coronary artery calcium score of 0 or more, coronary artery calcium score of 100 or more, coronary artery calcium score of 300 or more), and output a probability value for the probability that the coronary artery calcium score is 0 or more based on the calculated probability values.

In addition, the first diagnostic model (1100) may be trained using retinal images labeled with coronary artery calcium scores. The first diagnostic model (1100) may be trained based on not only retinal images labeled with a coronary artery calcium score of 0, but also all retinal images labeled with a coronary artery calcium score greater than 0. For example, the first diagnostic model (1100) may be trained on the remaining retinal images excluding duplicate retinal images, retinal images in which one of the binocular retinal images is missing, and low-quality retinal images from the retinal image data set labeled with coronary artery calcium scores. In addition, according to embodiments, retinal images of subjects in a specific age range (for example, under 40 years of age and 70 years of age or older) may be excluded from the training of the first diagnostic model (1100).

In addition, in one embodiment, the training target retinal images may be divided into a development set and an internal test set. For example, the development set and the internal test set may be randomly distributed at a ratio of 8:2. In addition, to prevent overfitting, training target retinal images may be classified into the development set or the internal test set by subject so that the retinal images of the same subject do not cross between the development set and the internal test set.

In addition, in one embodiment, the first diagnostic model (1100) may be trained using an Adam Optimizer with a schedule of 2e-4 learning rate, cosine learning rate, and 25 epochs. In addition, for data augmentation of the training target retinal images, at least one of mixup, cutmix, randaugment, enhancing contrast module, and random crop may be used. In addition, focal loss and exponential moving average may be used for training of the first diagnostic model (1100), and the size of the training target retinal image may be set to 384×384. In addition, to resolve the difficult label problem, for example, so that a coronary artery calcium score of 299 is not classified as 300, a sigmoid function is used for training of the first diagnostic model (1100), and soft labels may be used for training with coronary artery calcium score cutoffs of 100 and 300.

In addition, the processor of the diagnostic device may input the output value of the first diagnostic model (1100) and body information of the subject (at least one of age, sex, race, smoking status, blood pressure (for example, blood pressure level, presence or absence of hypertension), presence or absence of diabetes (or blood sugar level), cholesterol level) to the second diagnostic model (1200). Then, the processor of the diagnostic device may obtain a probability value and/or grade of the probability of occurrence of a cardiovascular disease-related event within 10 years of the subject from the second diagnostic model (1200). Here, the cardiovascular disease-related event within 10 years may include death due to cardiovascular disease, occurrence of cardiovascular disease, and occurrence of various events caused by cardiovascular disease (hospitalization, procedure, surgery, death, etc.) within 10 years from the time of taking the retinal image. In addition, in some cases, the processor of the diagnostic device may obtain a probability value of the probability of occurrence of a cardiovascular disease-related event within 10 years of the subject from the second diagnostic model (1200), and apply a predetermined cutoff value to the obtained probability value to obtain a grade corresponding to the obtained probability value.

The processor of the diagnostic device may output a probability value (score) and/or grade of the probability of occurrence of a cardiovascular disease-related event within 10 years of the subject as cardiovascular disease diagnostic information.

In addition, according to an embodiment, the processor of the diagnostic device may obtain a probability value and/or grade for the risk of current cardiovascular disease from the second diagnostic model (1200). In addition, according to an embodiment, the processor of the diagnostic device may obtain a probability value and/or grade of the probability of occurrence of a cardiovascular disease-related event within 10 years of the subject from the second diagnostic model (1200), and obtain a probability value and/or grade for the risk of current cardiovascular disease based on the obtained probability value and/or grade of the probability of occurrence of a cardiovascular disease-related event within 10 years of the subject. For example, the processor of the diagnostic device may compare the probability value and/or grade of the probability of occurrence of a cardiovascular disease-related event within 10 years of the subject obtained from the second diagnostic model (1200) with at least one or more predetermined reference probability values and/or grades, and obtain a probability value and/or grade for the risk of current cardiovascular disease based on the comparison result.

In addition, although the present specification describes focusing on cardiovascular disease-related events within 10 years, it is not limited thereto, and according to embodiments, cardiovascular disease-related events of various periods such as within 3 years, within 5 years, etc. may be applied to the description of the present specification.

In addition, in one embodiment, the second diagnostic model (1200) may be trained based on training target data. Here, the training of the second diagnostic model (1200) may include the meaning of fitting of the second diagnostic model (1200). In addition, the second diagnostic model (1200) may be configured using a regression-based Cox proportional hazards model.

As an example, the training target data may include body information of subjects of the training target retinal images of the first diagnostic model (1100), the results of follow-up observation of cardiovascular disease events over 10 years of the subjects, and probability values of the first diagnostic model (1100) for the training target retinal images. In addition, according to embodiments, the subjects of the training target retinal images of the first diagnostic model (1100) and the subjects of the training target data of the second diagnostic model (1200) may be the same, or at least partially different. For example, among the training target data of the subjects of the training target retinal images of the first diagnostic model (1100), training target data of subjects who have already experienced cardiovascular disease events or are suffering from cardiovascular disease at the reference time point may be excluded from the training of the second diagnostic model (1200). This is because by training the second diagnostic model (1200) using training target data for normal persons who have not experienced cardiovascular disease events and are not suffering from cardiovascular disease, the accuracy of the probability of occurrence of cardiovascular disease events within 10 years output from the second diagnostic model (1200) may be increased.

Then, through training of the second diagnostic model (1200), HR=−exp{β′} representing the hazard ratio may be estimated. Here, β′ may represent the regression coefficient. Then, Equation 1 below is derived through exponential transformation of the regression coefficient (β′) and the reference cumulative hazard function A0(t), and the second diagnostic model (1200) may obtain a probability value of the probability of occurrence of a cardiovascular disease-related event within 10 years of the subject based on Equation 1 below. For example, the Cox proportional hazards model of the second diagnostic model (1200) may be configured based on Equation 1.

$$CVD risk ( 10 \vert S ) = 1 - exp \{ - A 0 ( 10 ) exp \{ {β}^{′} S \times S + {β}^{′} Age \times Age + {β}^{′} Gender \times I ( Male ) \} \} \qquad [ Equation 1 ]$$

Here, CVD risk(10|S) represents the probability value of the probability of occurrence of a cardiovascular disease-related event within 10 years of the subject, A0(10) represents the reference cumulative function based on 10 years, S represents the probability value output from the first diagnostic model (1100) for the retinal image when the retinal image of the subject is input to the first diagnostic model (1100), AGE represents the age of the subject, and I(Male) represents the sex of the subject. For example, I(Male) may be an indicator function that reflects a value of 1 when the subject is male and reflects a value of 0 when the subject is female.

In addition, β′S may represent the regression coefficient for the output value output from the first diagnostic model (1100), β′Age may represent the regression coefficient for age, and β′gender may represent the regression coefficient for sex. Through training of the second diagnostic model (1200), the values of the regression coefficients may be adjusted.

In addition, the processor of the diagnostic device may obtain a probability value of the probability of occurrence of a cardiovascular disease-related event within 10 years from the second diagnostic model (1200), and apply a predetermined cutoff value to the probability value to obtain a grade corresponding to the probability value. Of course, in some cases, a predetermined cutoff value may be applied to the second diagnostic model (1200) in advance to obtain a grade corresponding to the probability value from the second diagnostic model (1200). At this time, the cutoff value may be one or more. For example, when there is one cutoff value, there may be two grades corresponding to the probability value, and when there are two cutoff values, there may be three grades corresponding to the probability value. The description of the present specification may be applied to various numbers of cutoff values and various cutoff values.

In addition, the processor of the diagnostic device may present the risk of cardiovascular disease at the 10th year or 5th year based on Equation 1 (or by applying Equation 1), and may present the risk of cardiovascular disease based on Equation 1 (or by applying Equation 1) for various groups of different sexes, different races, etc.

In one embodiment, the processor of the diagnostic device may set a predetermined cutoff value based on the population distribution of each group classified into a plurality of grades (that is, the incidence rate in each group) according to the follow-up observation result of a predetermined population. For example, the cutoff value may be determined based on the results of follow-up observation for a predetermined population. For example, based on the entirety of follow-up subjects, when follow-up observation results are obtained in which 0 to n1% is a low-risk group, n1 to n2% is a moderate-risk group, and n2 to 100% is a high-risk group, n1% and n2% may each be set as cutoff values. In addition, even if n1%, n2%, and n3% are not respectively cutoff values, the cutoff values may be set such that the population distribution in each risk group of the cardiovascular disease diagnostic information becomes similar to the follow-up observation result of a predetermined population.

In addition, the processor of the diagnostic device may set the cutoff value based on the incidence rate in cardiovascular disease biomarkers. For example, in the case of PCE, based on the PCE score, the risk of cardiovascular disease may be classified as low risk (PCE score 0% to 5%), borderline risk (5% to 7.4%), moderate risk (7.5% to 19.9%), and high risk (20% or more). And when the incidence rate in PCE, that is, the persons corresponding to low risk, borderline risk, moderate risk, and high risk is x1%, x2%, x3%, and x4% respectively, the cutoff value may be set so that the ratio of persons included in each grade of cardiovascular disease diagnostic information output from the processor of the diagnostic device is matched to x1%, x2%, x3%, and x4%. This may be applied not only to PCE but also to biomarkers such as QRISK3 and the modified Framingham risk score. In addition, the cutoff value may be a value optimized to best detect the group corresponding to high risk of each biomarker.

As a specific example, the processor of the diagnostic device may set the cutoff value using the ratio of high-risk groups and low-risk groups classified by a future 10-year cardiovascular risk calculator according to race or the 10-year cardiovascular risk calculation method of the applied clinical guideline. Accordingly, the performance of predicting the high-risk group of cardiovascular disease diagnostic information may be improved. For example, to maximize the performance of cardiovascular disease diagnostic information for diagnosing the high-risk group (intermediate & high) in PCE, the cutoff value of the cardiovascular disease diagnostic information may be set such that the ratio of all subjects corresponding to the low-risk group and the ratio of all subjects corresponding to the high-risk group of the cardiovascular disease diagnostic information become similar to the ratio of subjects corresponding to the low-risk group of PCE (for example, 53.1%) and the ratio of subjects corresponding to the high-risk group (for example, 46.9%). For example, the processor of the diagnostic device may set the cutoff value of cardiovascular disease diagnostic information such that the ratio of all subjects corresponding to the low-risk group and the ratio of all subjects corresponding to the high-risk group of cardiovascular disease diagnostic information are included within a predetermined range of the ratio of subjects corresponding to the low-risk group of PCE (for example, 53.1%) and the ratio of subjects corresponding to the high-risk group (for example, 46.9%). In addition, the processor of the diagnostic device may set the cutoff value of cardiovascular disease diagnostic information such that the ratio of all subjects corresponding to the low-risk group and the ratio of all subjects corresponding to the high-risk group of cardiovascular disease diagnostic information are included within a predetermined range of the ratio of subjects corresponding to a QRISK3 score of less than 10% (for example, 75.3%) and the ratio of subjects corresponding to a QRISK3 score of 10% or more (for example, 24.7%). In this case, the processor of the diagnostic device may accurately determine subjects with a QRISK3 score of 10% or more.

As such, while a biomarker of coronary artery calcium score is used in training of the first diagnostic model (1100), biomarkers different from the coronary artery calcium score, such as PCE, QRISK3, and the modified Framingham risk score, may be used in setting the cutoff value of the second diagnostic model (1200).

However, it is not limited thereto, and the coronary artery calcium score may be used in setting the cutoff value of the second diagnostic model (1200). For example, in the case of the coronary artery calcium score, based on the coronary artery calcium score, the risk of cardiovascular disease may be classified as low risk (coronary artery calcium score 0), moderate risk (coronary artery calcium score greater than 0 and less than or equal to 100), and high risk (coronary artery calcium score 100 or more). And when the incidence rate in the coronary artery calcium score, that is, the persons corresponding to low risk, moderate risk, and high risk is y1%, y2%, and y3% respectively, the cutoff value may be set so that the ratio of persons included in each grade of cardiovascular disease diagnostic information output from the processor of the diagnostic device is matched to y1%, y2%, and y3%. For example, in the above case, the processor of the diagnostic device may set the cutoff value of cardiovascular disease diagnostic information such that the ratio of all subjects corresponding to the low-risk group, the ratio of all subjects corresponding to the moderate-risk group, and the ratio of all subjects corresponding to the high-risk group of cardiovascular disease diagnostic information are included within a predetermined range of the ratio of subjects corresponding to the low-risk group of coronary artery calcium score (for example, y1%), the ratio of subjects corresponding to the moderate-risk group (for example, y2%), and the ratio of subjects corresponding to the high-risk group (for example, y3%).

In addition, in one embodiment, the processor of the diagnostic device may set the cutoff value of cardiovascular disease diagnostic information based on a specific ratio of subjects according to the coronary artery calcium score in a predetermined population. For example, the processor of the diagnostic device may output a first grade and a second grade as cardiovascular disease diagnostic information, and according to a specific ratio (for example, a1%) among the distribution of 0 to 100% of subjects according to the coronary artery calcium score, the processor of the diagnostic device may set the cutoff value of cardiovascular disease diagnostic information such that the ratio of all subjects corresponding to the first grade and the ratio of all subjects corresponding to the second grade of cardiovascular disease diagnostic information are matched to a % and 100%-a % (for example, such that the ratio of all subjects corresponding to the first grade and the ratio of all subjects corresponding to the second grade of cardiovascular disease diagnostic information are included in a predetermined range of the ratio of a % to 100%-a %).

In addition, when a cutoff value based on each biomarker is set, the grade output as cardiovascular disease diagnostic information from the processor of the diagnostic device may be similar to the actual result of the biomarker for which the cutoff value was set. For example, when a cutoff value according to the biomarker of PCE is applied to the probability value of the probability of occurrence of a cardiovascular disease-related event within 10 years of the subject output from the second diagnostic model (1200), and the grade of the subject is output from the processor of the diagnostic device, the grade of the subject output from the processor of the diagnostic device and the grade determined by the subject actually performing a test according to PCE may match. When an actual test is performed according to PCE, QRISK3, and modified Framingham risk score, inconvenience such as blood drawing of the subject may occur. However, in the case of the cardiovascular disease diagnosis method according to the present specification, since high-accuracy cardiovascular disease diagnostic information is obtained using only the retinal image of the subject, user convenience may be improved.

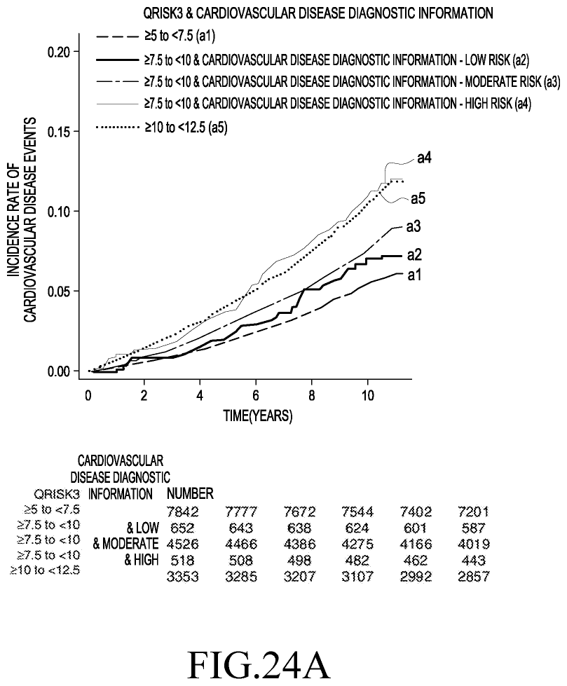

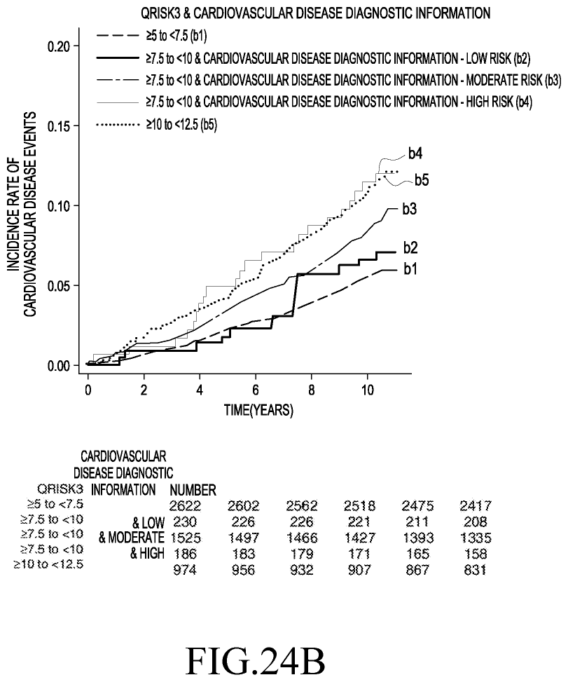

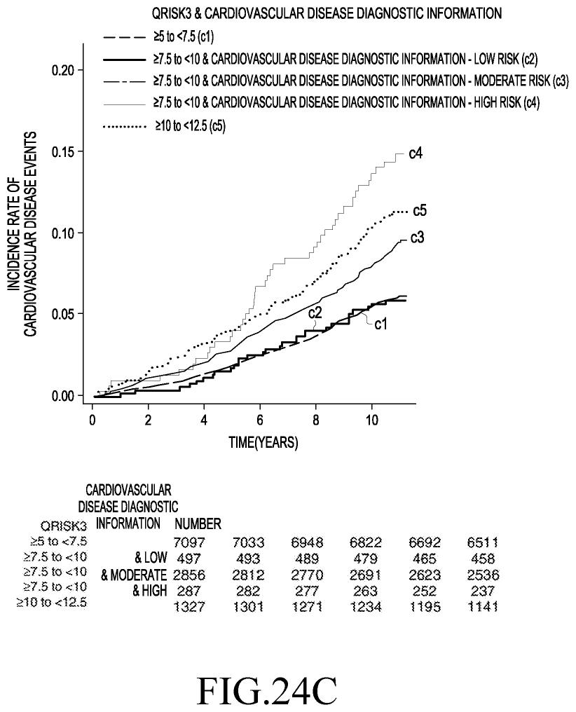

In addition, in one embodiment, when cardiovascular disease diagnostic information is applied to the score and/or grade of an existing biomarker, the incidence rate of cardiovascular disease-related events according to the score and/or grade of the existing biomarker may be classified or stratified according to each group (for example, low-risk group, moderate-risk group, and high-risk group) of the cardiovascular disease diagnostic information. This will be described in detail in the description of FIG. 24. And the cutoff of the cardiovascular disease diagnostic information may be set such that, when cardiovascular disease diagnostic information is applied to the score and/or grade of an existing biomarker, the incidence rate of cardiovascular disease-related events according to the score and/or grade of the existing biomarker is clearly stratified according to each group of the cardiovascular disease diagnostic information.

Hereinafter, embodiments of the cardiovascular disease diagnosis method according to the present specification will be described in detail.

### 2.1.3.1. EXAMPLE 1

Example 1 describes the experimental results of the cardiovascular disease diagnosis method according to the present specification using clinical data and retinal images of the UK Biobank. The UK Biobank is a prospective cohort of the United Kingdom.

In the experimental target data, among the clinical data and retinal images of the UK Biobank, the clinical data and retinal images of duplicate retinal images, low-quality retinal images, type 1 diabetes patients, persons already suffering from cardiovascular disease at the reference time point (specifically, persons suffering from heart disease, other heart disease, stroke, transient ischemic attack, peripheral artery disease, and persons who have undergone cardiovascular surgery or cardiovascular procedures), and persons under 40 years of age may be excluded from the experiment of Example 1. Accordingly, the experimental target data may be retinal images and data of 48,260 subjects.

In addition, the subjects of the experimental target data may be classified into three groups. The first group is persons not taking statins (45,473 persons), the second group is stage 1 hypertension patients not taking hypertension drugs (11,966 persons), and the third group may be middle-aged persons aged 40 to 64 at the reference time point (38,941 persons).

One of the objectives of the cardiovascular disease diagnosis method is for primary prevention of cardiovascular disease, and the experimental target data may be composed of data of normal persons (persons not having cardiovascular disease) who are highly likely to lack awareness of risk factors related to cardiovascular disease.

In one embodiment, QRISK3 scores for each subject of the experimental target data may be obtained. In addition, the subjects may be classified into 5 groups based on the QRISK3 score (0 to 5%, 5 to 10%, 10 to 15%, 15 to 20%, 20% or more). And since the recommended threshold for statin and hypertension drugs according to the guideline is a QRISK3 score of 10%, the group with a QRISK3 score of 7.5 to 10% may be additionally set as a borderline risk group.

In addition, the processor of the control device may input the experimental target data into the diagnostic model (1000). That is, the processor of the control device inputs the retinal data of the subjects into the first diagnostic model (1100) to obtain a probability value of the probability that the subject has a coronary artery calcium score of 0 or more from the first diagnostic model (1100), and inputs the probability value and body information of the subject (for example, age, sex) into the second diagnostic model (1200) to obtain a probability value of the probability of occurrence of a cardiovascular disease-related event within 10 years of each subject. In addition, the processor of the control device may classify the probability value using a predetermined cutoff value to classify the subjects into predetermined grades. In Example 1, to maximize the classification of cardiovascular disease occurrence risk and enable the application of cardiovascular disease diagnostic information compared to existing guidelines, the cutoff value may be set based on 40% and 95% of the population distribution of the scores of cardiovascular disease diagnostic information. As a result, such that the ratio of persons corresponding to the low-risk grade of cardiovascular disease diagnostic information is similar to the ratio of persons corresponding to the QRISK3 score of 0 to 5% (that is, the incidence rate corresponding to the QRISK3 score of 0 to 5%), 40% of the probability value of the probability of occurrence of a cardiovascular disease-related event within 10 years may be set as the first cutoff value. In addition, such that the ratio of persons corresponding to the moderate-risk grade of cardiovascular disease diagnostic information is similar to the ratio of persons corresponding to the QRISK3 score of 5 to 10% (that is, the incidence rate corresponding to the QRISK3 score of 5 to 10%), 95% of the probability value of the probability of occurrence of a cardiovascular disease-related event within 10 years may be set as the second cutoff value.

Hereinafter, the results according to Example 1 will be described. Hereinafter, the grade of cardiovascular disease diagnostic information may indicate the grade corresponding to the score of cardiovascular disease diagnostic information.

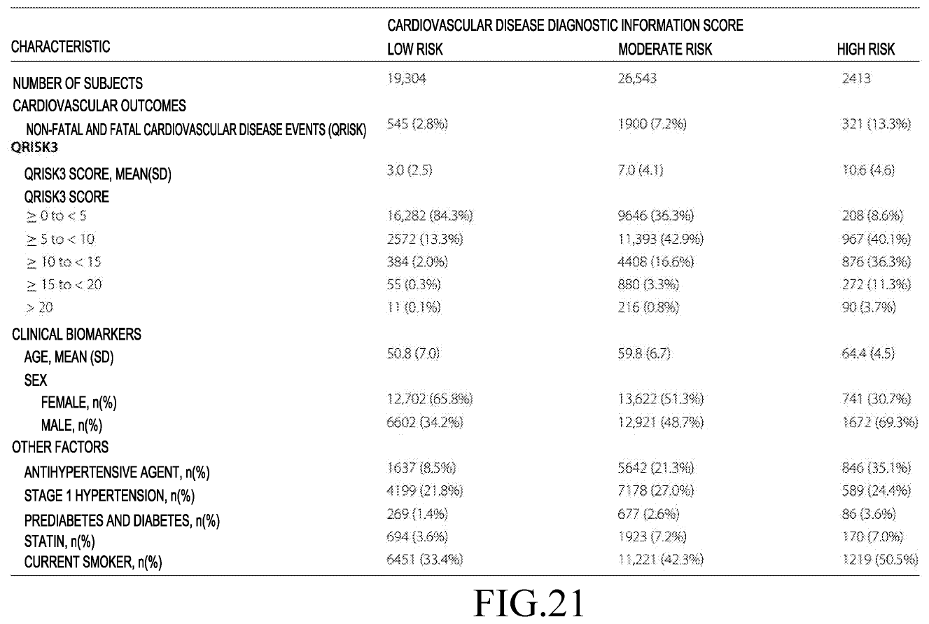

FIG. 21 shows clinical characteristics of subjects according to cardiovascular disease diagnostic information according to Example 1.

Referring to FIG. 21, among 48,260 subjects, the median of QRISK3's 10-year cardiovascular disease risk was 4.5% (IQR 2.2-8.0%, SD, 4.2%), and cardiovascular disease-related events occurred in 2,766 subjects (5.7%) during the follow-up period (up to 11.4 years). The Spearman's rank correlation coefficient between the probability value of cardiovascular disease diagnostic information and the QRISK3 score may be 0.50 (p<0.001).

In the low-risk grade of cardiovascular disease diagnostic information, the incidence rate of cardiovascular disease-related events was 2.8% (545/19,304), in the moderate-risk grade 7.2% (1,900/26,543), and in the high-risk group 13.3% (321/2413). Among the low-risk grade of cardiovascular disease diagnostic information, 84.3% had a QRISK3 score of 0 to less than 5%, and among the high-risk grade of cardiovascular disease diagnostic information, 8.6% had a QRISK3 score of 0 to less than 5%, 40.1% had a QRISK3 score of 5 to less than 10%, and 36.3% had a QRISK3 score of 10 to less than 15%.

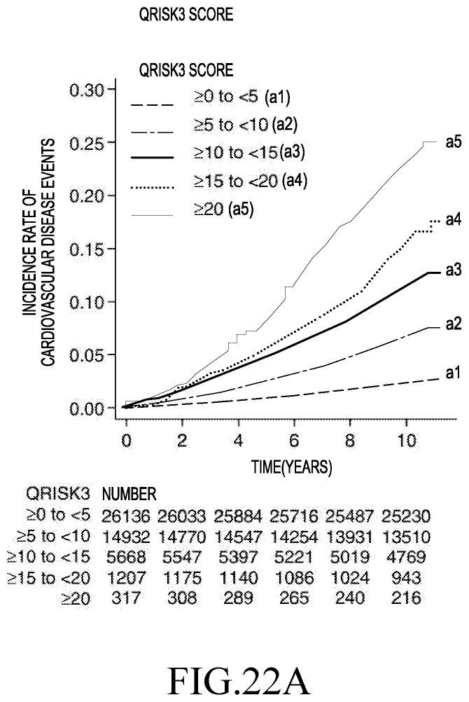

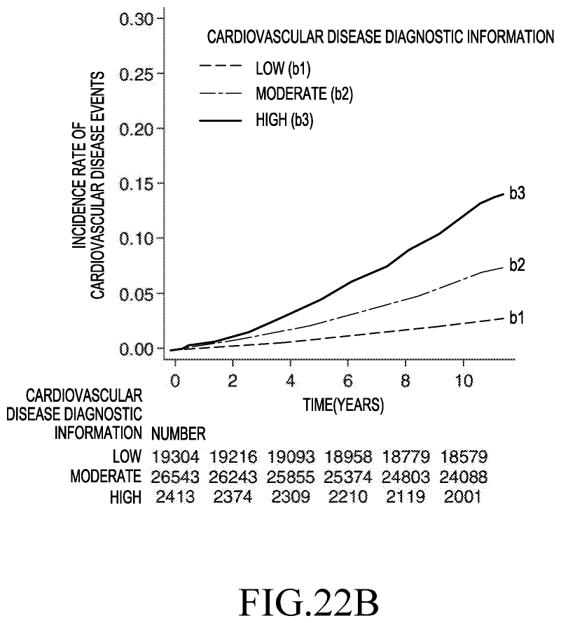

FIGS. 22a and 22b are diagrams for explaining incidence rates of cardiovascular disease-related events according to cardiovascular disease diagnostic information and QRISK3 according to Example 1.

Referring to FIGS. 22a and 22b, in the graphs of FIGS. 22a and 22b, the x-axis represents time (years) and the y-axis represents the incidence rate of cardiovascular disease-related events (in FIGS. 22a and 22b, expressed as cardiovascular disease event occurrence rate).

And the graph of FIG. 22a shows the incidence rate of cardiovascular disease-related events according to the QRISK3 score, and the graph of FIG. 22b shows the incidence rate of cardiovascular disease-related events according to cardiovascular disease diagnostic information. Specifically, the graph of FIG. 22a shows a Kaplan-Meier curve representing the result of performing Kaplan-Meier survival analysis on subjects of 5 groups according to the 5 grades of the QRISK3 score, and the graph of FIG. 22b may show a Kaplan-Meier curve representing the result of performing Kaplan-Meier survival analysis on subjects of 3 groups according to the 3 grades of cardiovascular disease diagnostic information.

As shown in the graph of FIG. 22a, QRISK3 can well distinguish cardiovascular disease risk in the general population of the UK Biobank. And as shown in the graph of FIG. 22b, cardiovascular disease diagnostic information can also clearly distinguish cardiovascular disease risk based on 3 groups within the general population of the UK Biobank. Based on cardiovascular disease diagnostic information, the probability of occurrence of cardiovascular disease-related events per 1000 person-years was 2.6 (95% confidence interval, 2.4-2.8) in the low-risk grade group, 6.8 (95% confidence interval, 6.5-7.1) in the moderate-risk grade group, and 13.1 (95% confidence interval, 11.7-14.6) in the high-risk grade group, and this may indicate that the probability of occurrence of cardiovascular disease-related events within 10 years in the high-risk group of cardiovascular disease diagnostic information is 13.1%. That is, as the cutoff value is set based on 40% and 95% of the population distribution of the scores of cardiovascular disease diagnostic information, the incidence rate of each group of cardiovascular disease diagnostic information and the incidence rate of each group in QRISK3 may become similar. In other words, the difference between the incidence rate of each group of cardiovascular disease diagnostic information and the incidence rate of each group in QRISK3 may be less than a predetermined threshold value. In addition, for persons not taking statins and stage 1 hypertension patients not taking hypertension drugs, similar tendencies as above may appear.

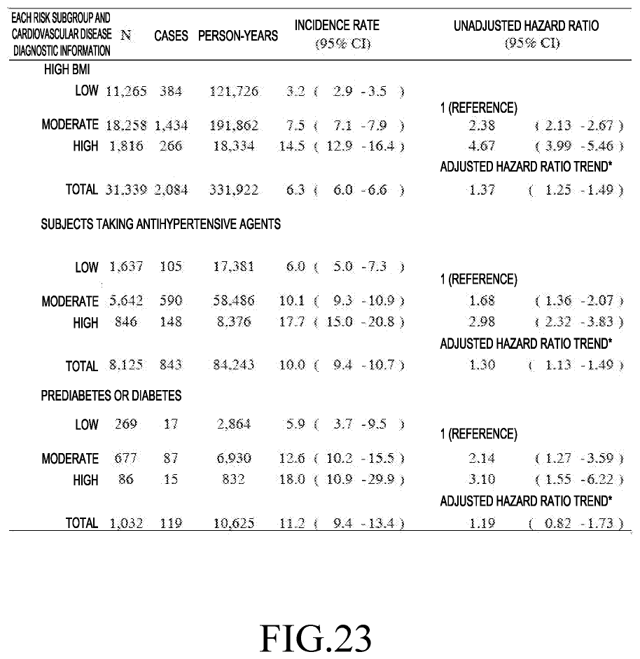

FIG. 23 is a diagram for explaining prediction performance of occurrence of cardiovascular disease-related events within 10 years of cardiovascular disease diagnostic information according to Example 1.

Referring to FIG. 23, the table of FIG. 23 may show the results of applying cardiovascular disease diagnostic information to a subgroup with high BMI (Body Mass Index) (BMI of 25 kg/m2 or more), a subgroup with hypertension (subjects taking antihypertensive drugs), and a subgroup with prediabetes or diabetes. In the table, CI represents the Confidence Interval, and N may represent the population of each group.

The results of the table of FIG. 23 may indicate that cardiovascular disease diagnostic information having 3 grades can additionally distinguish the risk of occurrence of cardiovascular disease in each of the above subgroups. In particular, in the subgroup with hypertension (subjects taking antihypertensive drugs), the incidence rate of cardiovascular disease-related events within 10 years in the group of high-risk grade of cardiovascular disease diagnostic information is 17.7 (95% confidence interval, 15.0-20.8), which is very high. When referring to FIGS. 22a, 22b, and 23, for normal persons not suffering from cardiovascular disease, persons not taking statins, stage 1 hypertension patients not taking hypertension drugs, persons with high BMI, persons taking hypertension drugs, and prediabetes/diabetes patients, the processor of the diagnostic device can predict with high accuracy the risk of cardiovascular disease (in particular, the risk of the probability of occurrence of cardiovascular disease-related events within 10 years being 10% or more) by providing cardiovascular disease diagnostic information based on a diagnostic model through input of retinal images and body information of the subject.

FIGS. 24a to 24c are diagrams for explaining results of applying cardiovascular disease diagnostic information to a group with a QRISK3 score of 7.5 to 10% according to Example 1.

Referring to FIG. 24, in the graphs of FIGS. 24a to 24c, the x-axis represents time (years) and the y-axis represents the incidence rate of cardiovascular disease-related events (in FIGS. 24a to 24c, expressed as cardiovascular disease event occurrence rate).

And the graph of FIG. 24a is for the group not taking statins, the graph of FIG. 24b is for the stage 1 hypertension group, and the graph of FIG. 24c is for the middle-aged (40 to 60 years of age) group, and each graph represents the result of performing Kaplan-Meier survival analysis.

In addition, in FIGS. 24a to 24c, a1, b1, and c1 represent the cardiovascular disease event occurrence rate of the group with a QRISK3 score of 5 to 7.5%, a2, b2, and c2 represent the cardiovascular disease event occurrence rate of the group with a QRISK3 score of 7.5 to 10% and with cardiovascular disease diagnostic information of a low-risk grade, a3, b3, and c3 represent the cardiovascular disease event occurrence rate of the group with a QRISK3 score of 7.5 to 10% and with cardiovascular disease diagnostic information of a moderate-risk grade, a4, b4, and c4 represent the cardiovascular disease event occurrence rate of the group with a QRISK3 score of 7.5 to 10% and with cardiovascular disease diagnostic information of a high-risk grade, and a5, b5, and c5 may represent the cardiovascular disease event occurrence rate of the group with a QRISK3 score of 10 to 12.5%.

Generally, the incidence rate of cardiovascular disease-related events in the group with a QRISK3 score of 10 to 12.5% may be higher than that of the group with a QRISK3 score of 7.5 to 10%. However, as shown in FIGS. 24a to 24c, when cardiovascular disease diagnostic information (for example, low-risk group, moderate-risk group, and high-risk group) is applied to the QRISK3 score, the incidence rate of cardiovascular disease-related events of one group according to the QRISK3 score may be classified or stratified according to each group of cardiovascular disease diagnostic information (for example, low-risk group, moderate-risk group, and high-risk group). For example, the incidence rate of cardiovascular disease-related events of the group with a QRISK3 score of 7.5 to 10% and with cardiovascular disease diagnostic information of a high-risk grade may tend to be higher than that of the group with a QRISK3 score of 10 to 12.5%. This may mean that the high-risk group that could not be predicted by the QRISK3 score, that is, the existing biomarker, can be accurately predicted by cardiovascular disease diagnostic information.

In addition, although the incidence rate of cardiovascular disease-related events of the group with a QRISK3 score of 7.5 to 10% should be higher than that of the group with a QRISK3 score of 5 to 7.5%, as cardiovascular disease diagnostic information is applied to the group with a QRISK3 score of 7.5 to 10% and the incidence rate of cardiovascular disease-related events is stratified, as shown in the graph of FIG. 24c, the cardiovascular disease event occurrence rates of the group with a QRISK3 score of 5 to 7.5% and the group with a QRISK3 score of 7.5 to 10% and with cardiovascular disease diagnostic information of a low-risk grade may show similar tendencies. This may mean that even for the borderline risk group still classified as a low-risk group for which drug treatment is not recommended with the QRISK3 score, that is, the existing biomarker, it is possible to further stratify and accurately predict that they belong to a lower or higher risk group using cardiovascular disease diagnostic information.

Therefore, the cardiovascular disease diagnostic information of the present specification can predict the risk of cardiovascular disease with high accuracy in the borderline risk group with a QRISK3 score of 7.5 to 10%, particularly more than QRISK3. Considering that the initiation of statin and antihypertensive drug treatment is recommended in the guideline when the QRISK3 score is 10% or more, this may mean that the cardiovascular disease diagnostic information of the present specification can serve as a Risk Enhancer that can find hidden risks in the borderline risk group with a QRISK3 score of 7.5 to 10% and serve as a guide for the initiation of statin and antihypertensive drug treatment.

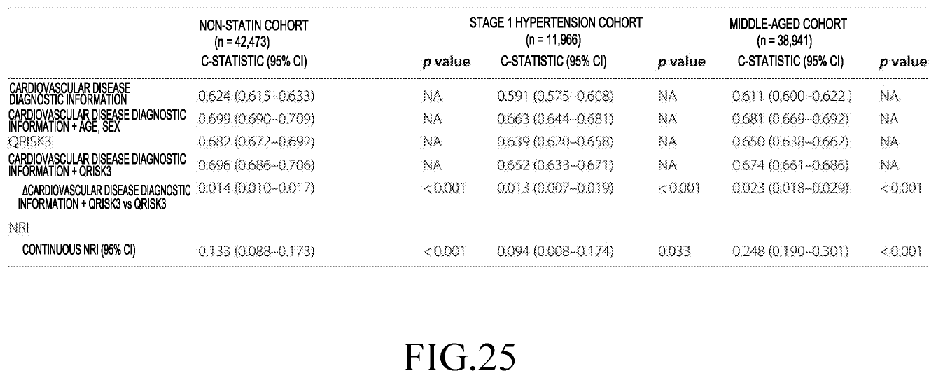

In addition, when the cardiovascular disease diagnostic information of the present specification and the QRISK3 score are applied together, the risk of cardiovascular disease can be predicted with high accuracy. This will be described in detail using FIG. 25.

FIG. 25 is a diagram for explaining in detail performance when cardiovascular disease diagnostic information and a QRISK3 score are applied together according to Example 1.

Referring to FIG. 25, in the table of FIG. 25, cardiovascular disease diagnostic information+age, sex represents the case where age and sex are added to the cardiovascular disease diagnostic information, cardiovascular disease diagnostic information+QRISK3 represents the case where the cardiovascular disease diagnostic information and QRISK3 are applied together, Δcardiovascular disease diagnostic information+QRISK3 vs QRISK3 represents the difference between the case where cardiovascular disease diagnostic information and QRISK3 are applied together and the case where only QRISK3 is applied, and NRI (Net reclassification Index) may represent an index that measures how much better a new model is than a past model. As shown in the table of FIG. 25, when cardiovascular disease diagnostic information and QRISK3 are applied together, the C statistic for the prediction of the occurrence of cardiovascular disease-related events increased by 0.014 (95% confidence interval, 0.010-0.017) in the group not taking statins, may increase by 0.013 (95% confidence interval, 0.007-0.019) in the stage 1 hypertension group, and by 0.023 (95% confidence interval, 0.018-0.029) in the middle-aged cohort. In addition, when cardiovascular disease diagnostic information and QRISK3 are applied together, the continuous NRI may be 0.133 (95% confidence interval, 0.088-0.173) in the cohort not taking statins (non-statin cohort), 0.094 (0.008-0.174) in the stage 1 hypertension cohort, and 0.248 (0.190-0.301) in the middle-aged cohort. From these results, it can be seen that when the cardiovascular disease diagnostic information of the present specification and the QRISK3 score are applied together, the risk of cardiovascular disease can be predicted with high accuracy.

In addition, in the table of FIG. 25, the C statistic for the prediction of the occurrence of cardiovascular disease-related events when age and sex are added to cardiovascular disease diagnostic information may appear to be higher than the C statistic for the prediction of the occurrence of cardiovascular disease-related events when only QRISK3 is used. Accordingly, it can be confirmed that the accuracy of predicting the risk of cardiovascular disease is higher when age and sex are added to cardiovascular disease diagnostic information than when only QRISK3 is used.

In addition, when the cardiovascular disease diagnostic information of the present specification is used or when the cardiovascular disease diagnostic information of the present specification and QRISK3 are applied together, more appropriate guide information may be provided to the subject. This will be described in detail below.

### 2.1.3.2. EXAMPLE 2

Example 2 describes the experimental results for the ability to identify individuals with moderate and high risk of cardiovascular disease diagnostic information according to the present specification using various biomarkers.

As described above, PCE, QRISK3, and the modified Framingham risk score may be official risk assessment tools and biomarkers used as guidelines for primary prevention of cardiovascular disease in the United States, the United Kingdom, and Singapore, respectively. However, these biomarkers are not non-invasive means that can distinguish moderate and high-risk groups of cardiovascular disease because blood tests are essential. On the other hand, the cardiovascular disease diagnosis method according to the present specification may be a retinal image-based biomarker that can non-invasively identify moderate and high-risk groups of cardiovascular disease. Hereinafter, it will be described that the cardiovascular disease diagnosis method according to the present specification can function as a non-invasive screening tool capable of identifying individuals according to the risk of cardiovascular disease, by comparing existing biomarkers and the cardiovascular disease diagnosis method according to the present specification.

In Example 2, data from the UK Biobank and SEED (Singapore Epidemiology of Eye Diseases) may be used as experimental target data. And in the entire data of the UK Biobank, clinical data and retinal images of duplicate retinal images, low-quality retinal images, type 1 diabetes patients, persons already suffering from cardiovascular disease at the reference time point (specifically, persons suffering from coronary artery disease, other heart disease, stroke, transient ischemic attack, peripheral artery disease, and persons who have undergone cardiovascular surgery or cardiovascular procedures), and persons under 40 years of age may be excluded from the experiment of Example 2. Accordingly, the clinical data and retinal images of 48,260 persons without a history of cardiovascular disease may be included in the experiment of Example 2.

In addition, in the entire data of SEED, clinical data and retinal images of persons with missing data and persons already suffering from cardiovascular disease at the reference time point may be excluded from the experiment of Example 2. And the clinical data and retinal images of 6,810 persons (2,548 Chinese, 1,976 Indian, 2,286 Malay) representing the general population without a history of cardiovascular disease in SEED may be included in the experiment of Example 2.

In addition, in Example 2, PCE scores, QRISK3 scores, and modified Framingham risk scores may be obtained for each subject of the experimental target data. Persons for whom cardiovascular disease risk management may be prioritized based on biomarkers such as PCE, QRISK3, and the modified Framingham risk score may be defined as moderate and high-risk groups. And persons for whom cardiovascular disease risk management may be prioritized based on biomarkers such as PCE, QRISK3, and the modified Framingham risk score may be defined as moderate and high-risk groups. For example, in the case of the PCE score, less than 7.5% is defined as a low-risk group, and 7.5% or more may be set as a moderate and high-risk group. In addition, in the case of the QRISK3 score and the modified Framingham risk score, less than 10% is defined as a low-risk group, and 10% or more may be set as a moderate and high-risk group.

In addition, in one embodiment, the experimental target data may be input into the diagnostic model (1000). That is, the processor of the control device inputs the retinal data of the subjects into the first diagnostic model (1100) to obtain a probability value of the probability that the subject has a coronary artery calcium score of 0 or more from the first diagnostic model (1100), and inputs the probability value and body information of the subject (for example, age, sex) into the second diagnostic model (1200) to obtain a probability value of the probability of occurrence of a cardiovascular disease-related event within 10 years of each subject. In addition, the processor of the control device may classify the probability value using a predetermined cutoff value to classify the subjects into predetermined grades.

In Example 2, the probability value may be classified using one cutoff value to output binary grades of moderate-high risk group and low-risk group. As an example, the cutoff value may be set such that the persons corresponding to the moderate-high risk group of cardiovascular disease diagnostic information are similar to the ratio of persons corresponding to 7.5% or more, which is the criterion of the moderate-high risk group of the PCE score (that is, the incidence rate corresponding to a PCE score of 7.5% or more). In addition, the cutoff value may be set such that the persons corresponding to the moderate-high risk group of cardiovascular disease diagnostic information are similar to the ratio of persons corresponding to 10% or more, which is the criterion of the moderate-high risk group of the QRISK3 score/modified Framingham risk score (that is, the incidence rate corresponding to a QRISK3 score/modified Framingham risk score of 10% or more).

In addition, as an example, the cutoff may be determined by performing preliminary analysis to achieve maximum performance of detection of the moderate-high risk group using the ROC (receiver operating characteristic) curve and the Youden Index.

Specifically, logistic regression may be performed using a set of continuous probability values of cardiovascular disease diagnostic information as the independent variable, and 1) binary categories of low-borderline risk group versus moderate-high risk group according to the PCE score of the UK Biobank, and 2) binary categories of low-risk group versus high-risk group according to the QRISK3 score of the UK Biobank and the modified Framingham risk score of SEED, respectively, as the dependent variable. According to such logistic regression, ROC curves according to the PCE score of the UK Biobank, the QRISK3 score of the UK Biobank, and the modified Framingham risk score of SEED may be generated, and the cutoff value may be determined by applying the Youden Index to the ROC curves. According to the determined cutoff value, the cardiovascular disease diagnosis method can identify moderate-high risk groups in each of PCE, QRISK3, and the modified Framingham risk score with high accuracy.

In addition, Covariate-adjusted ROC analysis may be performed to adjust potential covariates that affect cardiovascular disease risk in ROC analysis. Specifically, in traditional ROC analysis, the ROC curve may be defined as in Equation 2.

$$ROC ( u ) = P ( 1 - F 0 ( Y ) \leq u \vert D = 1 ) \qquad [ Equation 2 ]$$

Here, u represents the false positive rate, and Y may represent the predictor variable. F0(Y) represents the percentile value based on the marginal distribution among the control group, and ROC(u) may represent the sensitivity of the corresponding level.

In the case of Covariate-adjusted ROC analysis, instead of using the marginal distribution in traditional ROC analysis, the percentile value may be estimated based on the conditional distribution among the control group. To estimate the percentile under the conditional distribution, a linear model for the covariates of age and sex for Y may be regressed. According to such regression, the AUC (Area under covariate-adjusted ROC Curve) of the ROC curve according to each of the PCE score of the UK Biobank, the QRISK3 score of the UK Biobank, and the modified Framingham risk score of SEED may be improved (UK Biobank-PCE: 0.853 (95% CI: 0.849-0.859), UK Biobank-QRISK3: 0.820 (95% CI: 0.813-0.827), SEED-modified Framingham risk score_Chinese/Indian/Malay: 0.858 (95% CI: 0.832-0.879)/0.874 (95% CI: 0.848-0.892)/0.838 (95% CI: 0.794-0.87)). Accordingly, the variables of the second diagnostic model may be fitted (trained) and/or the cutoff may be determined to have higher accuracy.

Hereinafter, the results according to Example 2 will be described.

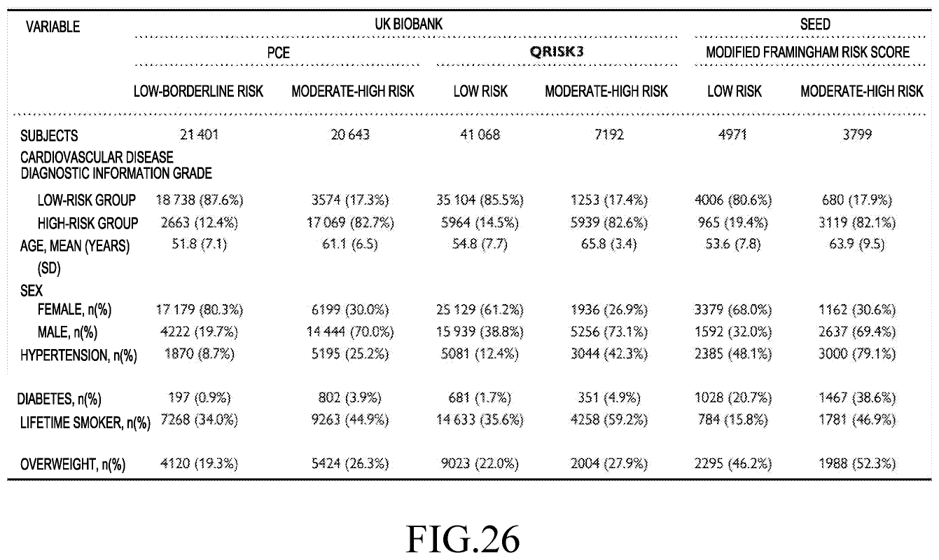

FIG. 26 shows clinical characteristics of subjects according to cardiovascular disease diagnostic information according to Example 2.

Referring to FIG. 26, the table of FIG. 26 shows the clinical characteristics of subjects of the UK Biobank and SEED, wherein each row represents subjects, the low-risk group and high-risk group according to cardiovascular disease diagnostic information, age, sex, hypertension, diabetes, smoking status, and overweight. And each column may represent the low-borderline risk group and the moderate-high risk group (cardiovascular disease risk rate within 10 years of 7.5% or more) according to the PCE score of the UK Biobank, and the low-risk group and the moderate-high risk group (cardiovascular disease risk rate within 10 years of 10% or more) according to the QRISK3 score of the UK Biobank and the modified Framingham risk score of SEED.

As shown in the table of FIG. 26, the ratio of persons belonging to the high-risk group according to cardiovascular disease diagnostic information among persons classified as the moderate-high risk group in each biomarker may all be 80% or more. Given these results, the cardiovascular disease diagnostic information of the present specification can predict with high accuracy persons classified as the moderate-high risk group in each biomarker.

In addition, for reference, as shown in the table of FIG. 26, cardiovascular disease-inducing factors such as age, sex, hypertension, diabetes, smoking status, and overweight may have a greater effect in the moderate-high risk group than in the low-risk group.

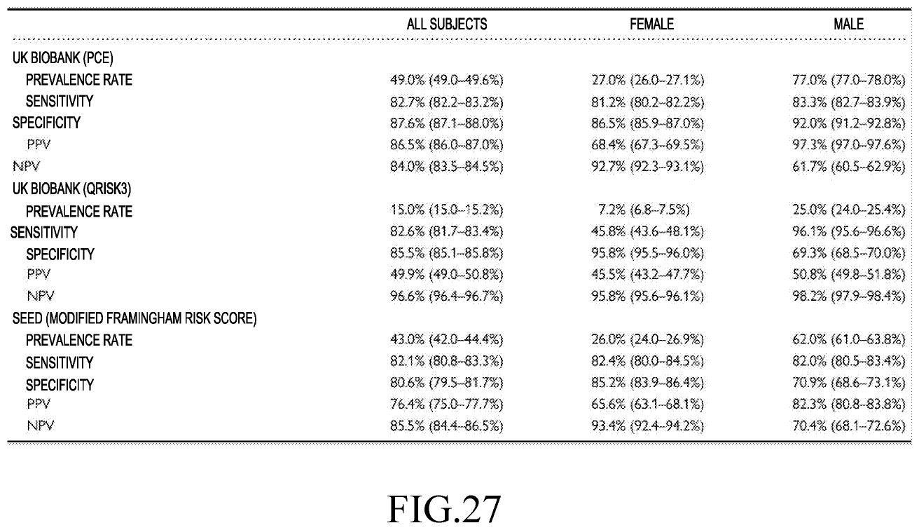

FIG. 27 is a diagram for explaining performance of cardiovascular disease diagnostic information according to Example 2.

Referring to FIG. 27, in the table of FIG. 27, each row represents the prevalence rate representing the overall ratio of subjects classified as the moderate-high risk group according to the PCE score of the UK Biobank, and the sensitivity, specificity, PPV (Positive Predictive Value), and NPV (Negative Predictive Value) of cardiovascular disease diagnostic information for the moderate-high risk group according to the PCE score of the UK Biobank. And the prevalence rate, sensitivity, specificity, PPV, and NPV are also described for the QRISK3 score of the UK Biobank and the modified Framingham risk score of SEED. And each column represents all subjects, female, and male.

As shown in the table of FIG. 27, cardiovascular disease diagnostic information can predict the moderate-high risk group according to the PCE score of the UK Biobank with 82.7% sensitivity, 87.6% specificity, 86.5% PPV, and 84.0% NPV. In addition, cardiovascular disease diagnostic information may predict the moderate-high risk group according to the QRISK3 score of the UK Biobank with 82.6% sensitivity, 85.5% specificity, 49.9% PPV, and 96.6% NPV, and may indicate the moderate-high risk group according to the modified Framingham risk score of SEED with 82.1% sensitivity, 80.6% specificity, 76.4% PPV, and 85.5% NPV.

As such, cardiovascular disease diagnostic information can predict with high accuracy the moderate-high risk group according to various biomarkers.

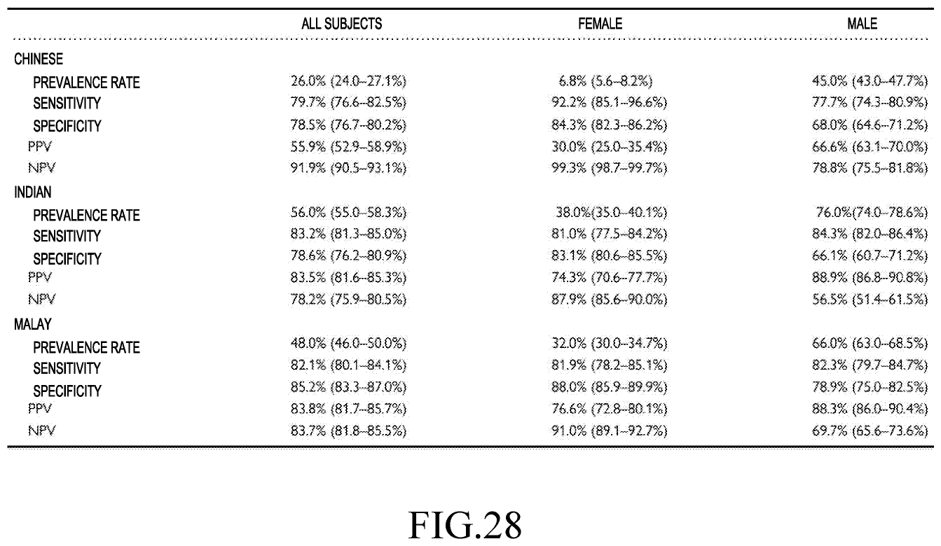

FIG. 28 is a diagram for explaining performance of cardiovascular disease diagnostic information with respect to various ethnicities according to Example 2.

Referring to FIG. 28, the table of FIG. 28 shows the performance of cardiovascular disease diagnostic information for the moderate-high risk group according to the modified Framingham risk score of SEED, wherein each row may represent the prevalence rate, sensitivity, specificity, PPV, and NPV for Chinese/Indian/Malay. And each column represents all subjects, female, and male.

As shown in the table of FIG. 28, cardiovascular disease diagnostic information can predict the moderate-high risk group according to the modified Framingham risk score of SEED for Chinese/Indian/Malay with high sensitivities of 79.7%, 83.2%, and 82.1%, respectively. In addition, cardiovascular disease diagnostic information can predict the moderate-high risk group according to the modified Framingham risk score of SEED for Chinese females with a very high NPV of 99.3%.

As such, cardiovascular disease diagnostic information can predict with high accuracy the moderate-high risk group according to other biomarkers for various ethnicities as well.

### 2.1.3.3. EXAMPLE 3

Example 3 is for verifying the performance of cardiovascular disease diagnostic information according to the present specification for the US population of AREDS (the Age-Related Eye Disease Studies).

In Example 3, data of subjects of AREDS may be used as experimental target data. In addition, the prediction performance of atherosclerotic cardiovascular disease (AtheroSclerotic CVD, ASCVD) of cardiovascular disease diagnostic information according to the present specification may be evaluated using the Cox proportional hazards model. At this time, the cardiovascular disease diagnostic information according to the present specification may be classified into three grades based on a predetermined cutoff value for the probability of occurrence of cardiovascular disease-related events within 10 years.

In Example 3, of 3,555 subjects, 282 (7.9%) experienced non-fatal and fatal ASCVD events, and 84 (2.4%) experienced fatal ASCVD during the 13-year follow-up period. In Example 3, as a result of evaluating whether adding cardiovascular diagnostic information to these improves risk prediction, cardiovascular diagnostic information was significantly associated with increased risk of ASCVD, the adjusted hazard ratio (HR) trend for non-fatal and fatal ASCVD events appears to be 1.13 (95% confidence interval, 1.03-1.24), and the adjusted HR trend for fatal ASCVD events appears to be 1.27 (1.07-1.52). Accordingly, cardiovascular diagnostic information greatly improved the overall prediction performance of the traditional risk model, the continuous NRI for non-fatal and fatal ASCVD events was 0.247 (0.106-0.364), and for fatal ASCVD events appears to be 0.232 (0.107-0.359). In addition, the processor of the diagnostic device can obtain a map for cardiovascular diagnostic information, and as a result of analyzing the map, it was confirmed that traditional risk features such as arteriovenous nicking and arterial narrowing were well detected by cardiovascular diagnostic information. Accordingly, the cardiovascular diagnostic information of the present specification may have significant potential as a biomarker or risk classification tool for cardiovascular disease applicable to the general US population.

### 2.1.3.4. EXAMPLE 4

Example 4 is for verifying the future cardiovascular risk prediction performance of the cardiovascular diagnosis method of the present specification based on the retrospective analysis of a prior prospective cohort study.

In Example 4, the main point may be verifying a 3-stage cardiovascular risk classification system using retinal images evaluated by cardiovascular disease diagnostic information. In Example 4, the cardiovascular disease diagnostic information may be based on the result output from the first diagnostic model and/or the result output from the second diagnostic model described above. And additionally, the coronary artery calcium score (CAC 0, >0-100, >100) measured by cardiac CT, the carotid intima-media thickness score (CIMT, <90% and ≥90%), and the brachial-ankle pulse wave velocity score (baPWV, <1800 and ≥1800 cm/s) may also be measured as independent variables for future cardiovascular disease diagnostic information. In addition, in Example 4, the cumulative incidence of non-fatal and/or fatal cardiovascular disease-related events was evaluated, and the hazard ratio (HR) trend was estimated using the Cox proportional hazards model.

As a result according to Example 4, in a predetermined clinical cohort (n=1106), 33 subjects (3.0%) experienced non-fatal or fatal cardiovascular disease-related events during 5 years, and cardiovascular disease diagnostic information was significantly associated with increased CVD risk (HR trend=2.02, 95% confidence interval, 1.26-3.24). In addition, in the multivariate Cox model including cardiovascular diagnostic information, coronary artery calcium score, carotid intima-media thickness score, brachial-ankle pulse wave velocity score, and other traditional risk factors, the probability value according to cardiovascular diagnostic information of the high-risk group may be very significantly associated with increased cardiovascular disease risk (HR=3.56 [1.34-9.51] at high risk when referring to low risk), while other biometric indicators may show lower association than cardiovascular diagnostic information. For example, the arteriosclerosis calcium score shows HR 2.45 [0.88-6.84] at more than 100 when referring to an arteriosclerosis calcium score of 0, the carotid intima-media thickness score shows HR 1.50 (0.64-3.51) at 90% or more compared to less than 90%, and the brachial-ankle pulse wave velocity score shows HR 1.27 (0.53-3.03) at 1800 cm/s or more compared to less than 1800 cm/s.

Accordingly, the cardiovascular disease diagnostic information of the present specification can predict cardiovascular disease risk with higher accuracy compared to the coronary artery calcium score, the carotid intima-media thickness score, and the brachial-ankle pulse wave velocity score.

### 2.1.4. Obtaining Information About Cardiovascular Disease Risk According to Existing Biomarkers

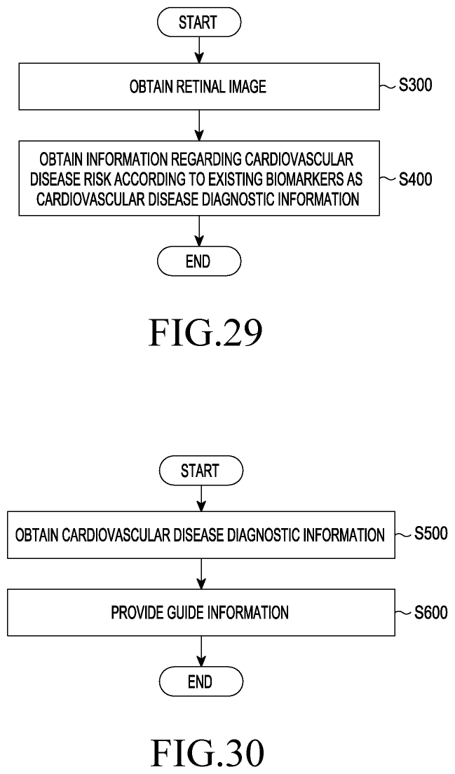

FIG. 29 is a diagram for explaining a cardiovascular disease diagnosis method according to another embodiment.

Referring to FIG. 29, the cardiovascular disease diagnosis method according to another embodiment may include a step of obtaining a retinal image (S300) and a step of obtaining information about cardiovascular disease risk according to existing biomarkers as cardiovascular disease diagnostic information (S400).

In step S300, the processor of the diagnostic device may obtain a retinal image. Since the contents described in FIG. 19 and above may be applied thereto, detailed description will be omitted.

In addition, in step S400, the processor of the diagnostic device may obtain information about cardiovascular disease risk according to existing biomarkers as cardiovascular disease diagnostic information. The contents described in the above-described diagnostic model may be applied to step S400.

Here, the information about cardiovascular disease risk according to existing biomarkers means the cardiovascular disease risk calculated according to existing biomarkers, and the processor of the diagnostic device may obtain information about the cardiovascular disease risk calculated according to existing biomarkers using retinal images. For example, according to step S400, the processor of the diagnostic device may obtain information about a coronary artery calcium score and/or a grade according to the coronary artery calcium score, a PCE score and/or a grade according to the PCE score, a QRISK3 score and/or a grade according to the QRISK3 score, a modified Framingham risk score and/or a grade according to the modified Framingham risk score, a carotid intima-media thickness score and/or a grade according to the carotid intima-media thickness score, and a brachial-ankle pulse wave velocity score and/or a grade according to the brachial-ankle pulse wave velocity score by using a diagnostic model based on a retinal image, and output the obtained information, or output information about a final score and/or a grade according to the final score based on the obtained information.

In one embodiment, the processor of the diagnostic device may obtain information about cardiovascular disease risk according to existing biomarkers as cardiovascular disease diagnostic information using a diagnostic model.

As an example, it may be learned using a retinal image and information about cardiovascular disease risk according to existing biomarkers labeled on the retinal image. For example, at least one of a coronary artery calcium score (and/or a grade according thereto), a PCE score (and/or a grade according thereto), a QRISK3 score (and/or a grade according thereto), a modified Framingham risk score (and/or a grade according thereto), a carotid intima-media thickness score (and/or a grade according thereto), or a brachial-ankle pulse wave velocity score (and/or a grade according thereto) may be labeled on each retinal image, and the diagnostic model may be learned using the retinal image and the label corresponding thereto. In addition, the label labeled on the retinal image may also include information about at least one of age, sex, race, smoking status, blood pressure (presence or absence of hypertension), and presence or absence of diabetes of the subject of the retinal image.

The processor of the diagnostic device may input the retinal image of a subject into a learned single diagnostic model to obtain information about cardiovascular disease risk according to existing biomarkers. For example, when the diagnostic model is learned based on the PCE score, the processor of the diagnostic device may input the retinal image into the diagnostic model, and obtain information about a PCE score and/or a grade according to the PCE score from the diagnostic model. In addition, according to an embodiment, the processor of the diagnostic device may also input information about at least one of age, sex, race, smoking status, blood pressure (presence or absence of hypertension), and presence or absence of diabetes of the subject together with the retinal image into the diagnostic imitation model, and obtain information about a PCE score and/or a grade according to the PCE score from the diagnostic model.

In addition, in one embodiment, the diagnostic models may be configured in parallel. For example, the description of the diagnostic model of 1.4.1.1 to 1.4.1.3 may be applied to the diagnostic model.

Specifically, the diagnostic model may include a plurality of diagnostic models. For example, the diagnostic model may include a first diagnostic model and a second diagnostic model. The first diagnostic model and the second diagnostic model may be learned using information about cardiovascular disease risk according to different biomarkers. For example, the first diagnostic model is learned using a retinal image labeled with a PCE score (and/or a grade according thereto), and the second diagnostic model may be learned using a retinal image labeled with a QRISK3 score (and/or a grade according thereto). Of course, it is not limited thereto, and additional diagnostic models such as a third/fourth diagnostic model may be included in the diagnostic model together with the first/second diagnostic model. In addition, the label labeled on the retinal image may also include information about at least one of age, sex, race, smoking status, blood pressure (presence or absence of hypertension), and presence or absence of diabetes of the subject of the retinal image.

In addition, the processor of the diagnostic device may input the retinal image of a subject into the first diagnostic model and the second diagnostic model to obtain information about cardiovascular disease risk according to different biomarkers. For example, the processor of the diagnostic device may obtain a PCE score (and/or a grade according thereto) of the subject from the first diagnostic model, obtain a QRISK3 score (and/or a grade according thereto) of the subject from the second diagnostic model, and output the obtained information. In addition, the processor of the diagnostic device may output information about a final score and/or a grade according to the final score based on the information obtained from each diagnostic model. For example, based on the PCE score (and/or a grade according thereto) of the subject obtained from the first diagnostic model and the QRISK3 score (and/or a grade according thereto) of the subject obtained from the second diagnostic model, information about a final score and/or a grade according to the final score may be obtained and output. Accordingly, the processor of the diagnostic device may obtain information about cardiovascular disease risk according to existing biomarkers as cardiovascular disease diagnostic information non-invasively using retinal images, without using invasive methods such as blood tests.

In addition, in one embodiment, the diagnostic models may be configured in series. For example, the description of the diagnostic models of 1.4.2.1, 1.4.2.2, and 2.1.2 may be applied to the diagnostic model.

As an example, the diagnostic model may include a first diagnostic model and a second diagnostic model as in FIG. 20. For example, the processor of the diagnostic device may input a retinal image of a subject to the first diagnostic model, and obtain a probability value of the probability that the subject has a coronary artery calcium score of 0 or more from the first diagnostic model. In addition, the processor of the diagnostic device may input the output value of the first diagnostic model and body information of the subject (at least one of height, weight, age, sex, race, smoking status, blood pressure (for example, blood pressure level, presence or absence of hypertension), presence or absence of diabetes (or blood sugar level), cholesterol level). At this time, similarly to Example 2, the second diagnostic model may be set with cutoff values based on various biomarkers such as PCE, QRISK3, and the modified Framingham risk score. For example, as the cutoff value of the second diagnostic model, a cutoff value set to be similar to the ratio of persons corresponding to 7.5% or more, which is the criterion of the moderate-high risk group of the PCE score, and/or a cutoff value set to be similar to the ratio of persons corresponding to 10% or more, which is the criterion of the moderate-high risk group of the QRISK3/modified Framingham risk score, may be set. Accordingly, cardiovascular disease diagnostic information can predict with high accuracy the moderate-high risk group according to various biomarkers. For obtaining information about cardiovascular disease risk according to existing biomarkers when the diagnostic models are configured in series, the contents described in the above-described Example 2 may be applied.

### 2.1.5. Providing Guide Information for Cardiovascular Disease Diagnostic Information

FIG. 30 is a diagram for explaining a method for providing guide information for cardiovascular disease diagnostic information according to one embodiment.

Referring to FIG. 30, the guide information providing method according to one embodiment may include a step of obtaining cardiovascular disease diagnostic information (S500) and a step of providing guide information for the diagnostic information (S600).

In step S500, the processor of the diagnostic device may obtain diagnostic information. Since the description above may be applied to step S500, detailed description will be omitted.

In addition, in step S600, the processor of the diagnostic device may provide guide information for the diagnostic information. Although steps S500 and S600 are described focusing on cardiovascular disease for convenience of description, it is not limited thereto, and the guide information of the present specification may of course also be applied to various diseases such as ophthalmic disease, cardiovascular disease, and renal disease. Accordingly, steps S500 and S600 will be described focusing on guide information according to cardiovascular diagnostic information.

In one embodiment, the guide information may mean information about medical/non-medical treatments recommended to the subject according to the cardiovascular disease diagnostic information. As an example, the guide information may include prescription information, measure information, and management information.

The prescription information may mean information about drugs (for example, ethical drugs) recommended to the subject in order to maintain or improve the cardiovascular disease risk according to the cardiovascular disease diagnostic information. In addition, the prescription information may include information about the drugs to be prescribed, the time of taking, and the amount of taking. For example, the prescription information may include information about a prescription of one or more of HMG-CoA reductase inhibitor statin (including various formulations such as simvastatin, atorvastatin, rosuvastatin, etc.) series drugs, PCSK9 inhibitor, fibric acid derivative combination therapy, aspirin, bile acid sequestrant, nicotinic acid, Omega-3 fatty acid, ezetimibe, and fibrate.

In addition, the measure information may mean information about future measures recommended to the subject in order to maintain or improve the cardiovascular disease risk according to the cardiovascular disease diagnostic information. For example, the additional examination information may include information about secondary diagnosis or medical treatment of the subject. As an example, the additional examination information may include information about additionally required examinations, information about hospitals/medical staff capable of additional examinations, and information about recommended procedures/surgeries.

In addition, the management information may include information about non-medical treatments recommended to the subject in order to maintain or improve the cardiovascular disease risk according to the cardiovascular disease diagnostic information. For example, the management information may include information about lifestyle habits, dietary habits, exercise, and non-prescription drugs such as nutritional supplements for lowering the risk of cardiovascular disease.

In addition, in one embodiment, the diagnostic device may be linked with an external monitoring device. Here, the monitoring device may mean a device that monitors the lifestyle or behavior of the subject. For example, the monitoring device may include portable devices, wearable devices, wellness measurement devices, etc. In addition, the monitoring device may be the above-described client device. For example, the monitoring device includes an imaging unit, and may obtain an image of the inside and outside of the eye by photographing the inside and outside of the eye through the imaging unit.

And the monitoring device may monitor various information such as the activity level, exercise method, exercise time, food intake, intake amount, health supplement intake information, sleep time, sleep habit, heart rate, blood pressure, blood sugar level, body water content, oxygen level, body temperature, oxygen saturation, pulse wave, hospital visit, whether examination was performed, whether procedure/surgery was performed, ocular image of the subject.

The processor of the diagnostic device may perform communication with the monitoring device by wire or wirelessly through the communication module.

The processor of the diagnostic device may provide the guide information to the monitoring device. And the monitoring device may provide various information to the subject based on the guide information and the monitored information. For example, the monitoring device obtains management information (for example, lifestyle information, dietary habit information, exercise information) as guide information from the diagnostic device, compares the management information with the monitored information to determine whether the monitored information matches the management information, and may provide the determination result and/or additional information according thereto.

For example, when the monitored exercise time is less than the exercise time of the management information, the monitoring device may provide information to the subject to exercise according to the management information. In addition, when the food intake amount among the monitored information corresponds to the food intake amount of the management information, the monitoring device may provide information to the subject that food intake is being done well according to the management information.

In addition, the processor of the diagnostic device may obtain the monitored information from the monitoring device. The processor of the diagnostic device may provide various information to the subject based on the guide information and the monitored information. For example, the processor of the diagnostic device may compare the monitored information with the guide information to determine whether the monitored information matches the management information, and provide the determination result and/or additional information according thereto. The example of the monitoring device described above may be applied to the operation of the processor of the diagnostic device.

In addition, the processor of the diagnostic device may generate guide information by reflecting the monitoring information received from the monitoring device. For example, the processor of the diagnostic device may obtain state information (exercise state, life state, dietary habit state, etc.) of the subject based on the monitoring information, and modify guide information determined as cardiovascular disease diagnostic information to suit the subject based on the state information of the subject.

In one embodiment, the processor of the diagnostic device may provide guide information using a predetermined database. For example, the diagnostic device may include a database that matches the score and/or grade of cardiovascular disease diagnostic information with guide information. For example, when the cardiovascular disease diagnostic information is expressed in 3 grades, guide information matching the low-risk grade (for example, prescription information—none, measure information—information on the next treatment time, management information—provision of dietary habit information, provision of exercise information), guide information matching the moderate-risk grade (for example, prescription information—none, measure information—provision of additional examination information, management information—provision of dietary habit information, provision of exercise information, provision of non-prescription drug information), and guide information matching the high-risk grade (for example, prescription information—provision of statin prescription information, measure information—additional examination information, provision of recommended procedure/surgery information, management information—provision of dietary habit information, provision of exercise information, provision of non-prescription drug information) may be included in the database. The processor of the diagnostic device may provide guide information matching the cardiovascular disease diagnostic information based on the database.

In addition, in another embodiment, the processor of the diagnostic device may provide guide information using a machine learning model. For example, the diagnostic device may include a guide information model based on a machine learning model or a neural network model. The guide information model may be learned based on the score and/or grade of cardiovascular disease diagnostic information and guide information. In addition, additionally, the guide information model may also be learned together with body information of the subject (at least one of height, weight, age, sex, race, smoking status, blood pressure (for example, blood pressure level, presence or absence of hypertension), presence or absence of diabetes (or blood sugar level), cholesterol level). Accordingly, the processor of the diagnostic device may obtain guide information about the subject by inputting the score and/or grade of cardiovascular disease diagnostic information and the body information of the subject into the guide information model. In addition, according to embodiments, the guide information model may be included in the diagnostic model, or may be configured independently of the diagnostic model.

### 2.1.6. Providing Guide Information Using Results According to Existing Biomarkers and Cardiovascular Disease Diagnostic Information

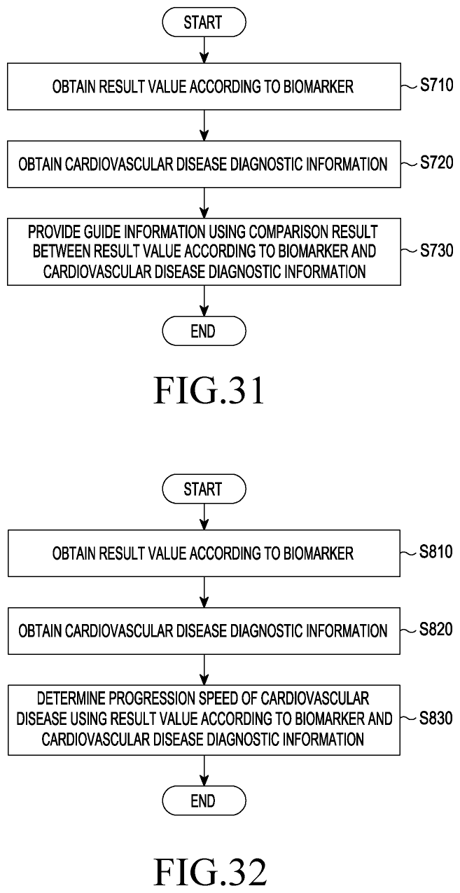

FIG. 31 is a diagram for explaining a method for providing guide information using results according to existing biomarkers and cardiovascular disease diagnostic information according to one embodiment.

Referring to FIG. 31, the guide information providing method according to one embodiment may include a step of obtaining a result value according to a biomarker (S710), a step of obtaining cardiovascular disease diagnostic information (S720), and a step of providing guide information using a comparison result between the result value according to the biomarker and cardiovascular disease diagnostic information (S730).

According to step S710, the processor of the diagnostic device may obtain a result value according to a biomarker. For example, the processor of the diagnostic device may obtain the score and/or grade of a biomarker from an external device or the input module of the diagnostic device. Here, the biomarker may include the above-described PCE, QRISK, modified Framingham risk score, carotid intima-media thickness, brachial-ankle pulse wave velocity, etc. In addition, the biomarker may include other cardiovascular disease biomarkers or risk assessment tools for cardiovascular disease. For example, the processor of the diagnostic device may also obtain information about blood pressure rise rate, LDL rise rate, HbA1c rise rate, and frequency of uncontrolled diabetes/hypertension/hyperlipidemia (for example, blood pressure of 160 mmHg or more observed 4 times in the past 6 months, etc.).

In addition, in step S720, the processor of the diagnostic device may obtain cardiovascular disease diagnostic information. Here, the cardiovascular disease diagnostic information may mean cardiovascular disease diagnostic information based on the retinal image of the same subject as the subject of the result value according to the biomarker obtained in step S710. Since the description above may be applied to step S720, detailed description will be omitted.

In addition, in step S730, guide information may be provided using a comparison result between the result value according to the biomarker and cardiovascular disease diagnostic information.

Generally, medical guidelines specifying the risk of cardiovascular disease and medical treatment information accordingly are used in the medical community. And the biomarkers for determining the risk of cardiovascular disease may be different in each country. For example, QRISK3 is used in the United Kingdom, PCE is used in the United States, and the modified Framingham risk score may be used in Singapore.

Hereinafter, an embodiment of providing guide information using a comparison result between the result value according to the biomarker included in the medical guidelines and cardiovascular disease diagnostic information will be described.

In the embodiment, when the biomarker included in the medical guideline is QRISK3, the processor of the diagnostic device may compare the score or grade of QRISK3 with the score or grade according to cardiovascular disease diagnostic information.

For example, when the result according to QRISK3 is a high-risk group and the result according to cardiovascular disease diagnostic information is a high-risk grade, the processor of the diagnostic device may determine that the cardiovascular disease risk of the subject is high risk, and provide treatment information for the high-risk group in the medical guideline. In addition, according to an embodiment, the processor of the diagnostic device may provide the guide information provided when the cardiovascular disease diagnostic information is determined to be a high-risk group described in FIG. 30 (for example, prescription information—provision of statin prescription information, measure information—additional examination information, provision of recommended procedure/surgery information, management information—provision of dietary habit information, provision of exercise information, provision of non-prescription drug information).

In addition, when the result according to QRISK3 is a high-risk group and the result according to cardiovascular disease diagnostic information is a non-high-risk grade (for example, low-risk grade or moderate/borderline risk grade), the processor of the diagnostic device may determine that the cardiovascular disease risk of the subject is high risk, and provide treatment information for the high-risk group in the medical guideline. In addition, according to an embodiment, the processor of the diagnostic device may also provide the guide information provided when the cardiovascular disease diagnostic information is determined to be a high-risk group.

In addition, when the result according to QRISK3 is a non-high-risk group (for example, low-risk group or moderate/borderline risk grade) and the result according to cardiovascular disease diagnostic information is a high-risk grade, the processor of the diagnostic device may determine that the cardiovascular disease risk of the subject is a high-risk group even if the result according to QRISK3 is a non-high-risk group. This may be based on the experimental result showing that, as described in Example 1 above, even if determined to be a non-high-risk group by QRISK3, the high-risk group that could not be predicted by QRISK3 can be accurately predicted by cardiovascular disease diagnostic information. Accordingly, the processor of the diagnostic device may provide the guide information provided when the cardiovascular disease diagnostic information is determined to be a high-risk group.

In addition, when the result according to QRISK3 is a non-high-risk group and the result according to cardiovascular disease diagnostic information is a non-high-risk grade, the processor of the diagnostic device may determine the cardiovascular disease risk of the subject as a non-high-risk group. Accordingly, the processor of the diagnostic device may provide the guide information provided when the cardiovascular disease diagnostic information is determined to be a non-high-risk group (for example, prescription information—none, measure information—information on the next treatment time, management information—provision of dietary habit information, provision of exercise information).

In addition, hereinafter, an embodiment of providing guide information using a comparison result between the result value according to the biomarker not included in the medical guideline and cardiovascular disease diagnostic information will be described.

In the embodiment, when the biomarker not included in the medical guideline but used for reference is QRISK3, the processor of the diagnostic device may compare the score or grade of QRISK3 with the score or grade according to cardiovascular disease diagnostic information.

For example, when the result according to QRISK3 is a high-risk group and the result according to cardiovascular disease diagnostic information is a high-risk grade, the processor of the diagnostic device may determine that the cardiovascular disease risk of the subject is high risk, and provide the guide information provided when the cardiovascular disease diagnostic information is determined to be a high-risk group.

In addition, when the result according to QRISK3 is a high-risk group and the result according to cardiovascular disease diagnostic information is a non-high-risk grade, the processor of the diagnostic device may determine the cardiovascular disease risk of the subject as a non-high-risk group. This may be based on the experimental result (in particular, the embodiment of FIG. 24c) showing that, as described in Example 1 above, even if determined to be a somewhat high-risk group by QRISK3, it can be accurately predicted using cardiovascular disease diagnostic information that they belong to a lower risk group. Accordingly, the processor of the diagnostic device may provide the guide information provided when the cardiovascular disease diagnostic information is determined to be a non-high-risk group. In addition, according to an embodiment, when the result according to QRISK3 is a high-risk group and the result according to cardiovascular disease diagnostic information is a non-high-risk grade, the processor of the diagnostic device may determine that the cardiovascular disease risk of the subject is high risk for preventive purposes, and provide the guide information provided when the cardiovascular disease diagnostic information is determined to be a high-risk group.

In addition, when the result according to QRISK3 is a non-high-risk group (for example, low-risk group or moderate/borderline risk grade) and the result according to cardiovascular disease diagnostic information is a high-risk grade, the processor of the diagnostic device may determine the cardiovascular disease risk of the subject as a high-risk group even if the result according to QRISK3 is a non-high-risk group. This may be based on the experimental result (in particular, the embodiment of FIG. 24) showing that, as described in Example 1 above, even if QRISK3 is determined to be a non-high-risk group, the high-risk group that could not be predicted by QRISK3 can be accurately predicted by cardiovascular disease diagnostic information. Accordingly, the processor of the diagnostic device may provide the guide information provided when the cardiovascular disease diagnostic information is determined to be a high-risk group.

In addition, when the result according to QRISK3 is a non-high-risk group and the result according to cardiovascular disease diagnostic information is a non-high-risk grade, the processor of the diagnostic device may determine the cardiovascular disease risk of the subject as a non-high-risk group, and provide the guide information provided when the cardiovascular disease diagnostic information is determined to be a non-high-risk group.

In the above embodiments, the results according to the biomarkers and the results according to cardiovascular disease diagnostic information were described with two grades of high risk and non-high risk, but it is not limited thereto, and the contents described above may be applied even when the results according to the biomarkers and the results according to cardiovascular disease diagnostic information are of three or more grades.

### 2.1.7. Prediction of Cardiovascular Disease Progression Speed Using Cardiovascular Disease Diagnostic Information

FIG. 32 is a diagram for explaining a method for predicting a cardiovascular disease progression speed using cardiovascular disease diagnostic information according to one embodiment.

Referring to FIG. 32, the cardiovascular disease progression speed prediction method according to one embodiment may include a step of obtaining a result value according to a biomarker (S810), a step of obtaining cardiovascular disease diagnostic information (S820), and a step of determining the progression speed of cardiovascular disease using the result value according to the biomarker and the cardiovascular disease diagnostic information (S830).

Since the matters described in the above-described steps S710 and S720 may be applied to steps S810 and S820, detailed description will be omitted.

In addition, in step S830, the progression speed of cardiovascular disease may be determined using the result value according to the biomarker and the cardiovascular disease diagnostic information.

Specifically, even if the result values according to the biomarker are the same, the progression speed of cardiovascular disease may be different. For example, even if the QRISK3 score is 8% which is a low-risk group, the score of cardiovascular disease diagnostic information may be different. However, even if the QRISK3 score is the same, a person with a high score of cardiovascular disease diagnostic information may have a faster progression speed of cardiovascular disease than a person with a low score of cardiovascular disease diagnostic information.

In one embodiment, the processor of the diagnostic device may determine whether the score of cardiovascular disease diagnostic information is equal to or greater than a predetermined threshold value. And when the score of cardiovascular disease diagnostic information is equal to or greater than the predetermined threshold value, it may be determined that the progression speed of cardiovascular disease is fast, and when the score of cardiovascular disease diagnostic information is less than the predetermined threshold value, it may be determined that the progression speed of cardiovascular disease is slow.

In addition, in one embodiment, the processor of the diagnostic device may predict the cardiovascular disease progression speed based on the change amount of cardiovascular disease diagnostic information.

For example, the processor of the diagnostic device may obtain first cardiovascular disease diagnostic information and second cardiovascular disease diagnostic information of a subject. Here, the first cardiovascular disease diagnostic information and the second cardiovascular disease diagnostic information may be based on retinal images of the same subject taken at different times. For example, the first cardiovascular disease diagnostic information may be based on a retinal image taken at an earlier time than the second cardiovascular disease diagnostic information.

In addition, the processor of the diagnostic device may obtain the change amount of the obtained cardiovascular disease diagnostic information. For example, the processor of the diagnostic device may obtain the change amount of cardiovascular disease diagnostic information based on the time difference between the time when the retinal image of the first cardiovascular diagnostic information was taken and the time when the retinal image of the second cardiovascular diagnostic information was taken, and the score difference between the score of the first cardiovascular diagnostic information and the score of the second cardiovascular diagnostic information.

In addition, the processor of the diagnostic device may predict the cardiovascular disease progression speed of the subject based on the change amount of cardiovascular disease diagnostic information. For example, the processor of the diagnostic device may determine that the cardiovascular disease progression speed is fast when the change amount of cardiovascular disease diagnostic information is equal to or greater than a predetermined threshold value, and determine that the cardiovascular disease progression speed is slow when the change amount of cardiovascular disease diagnostic information is less than the predetermined threshold value. Such threshold values may be set to one or more. In addition, according to embodiments, the threshold value may be set by various criteria. For example, the threshold value may be set such that the incidence rate of cardiovascular disease occurrence events is clearly stratified.

In addition, the processor of the diagnostic device may provide information about the cardiovascular disease progression speed and/or guide information according thereto. In this case, as the cardiovascular disease progression speeds of the first subject and the second subject are different, the processor of the diagnostic device may provide different guide information to the first subject and the second subject.

For example, even when the first subject and the second subject belong to the same risk group according to existing biomarkers and/or cardiovascular disease diagnostic information, different guide information may be provided according to the cardiovascular disease progression speeds of the first subject and the second subject. For example, when the cardiovascular disease progression speed of the first subject is predicted to be fast and the first subject is in a low-risk group, the processor of the diagnostic device may provide guide information to the effect that management of blood pressure, diabetes, hypertension, etc. should be thoroughly performed. As an example, the processor of the diagnostic device may provide measure information including information on additional examinations (for example, description of additional examinations, additional examination date, information on hospitals/medical staff capable of additional examinations, etc.) and/or management information including recommended lifestyle correction goals, recommended dietary habit information, etc., as guide information.

In addition, when the cardiovascular disease progression speed of the first subject is predicted to be fast and the first subject is in a high-risk group, the processor of the diagnostic device may increase the prescription amount of the first subject as prescription information.

In addition, the processor of the diagnostic device may perform communication with the above-described monitoring device. The processor of the diagnostic device may provide guide information determined according to the cardiovascular disease progression speed to the monitoring device. For example, when the processor of the diagnostic device determines that the cardiovascular disease progression speed of the first subject of the low-risk group of cardiovascular disease diagnostic information who has no symptoms and does not have cardiovascular risk factors is fast, measure information according thereto (for example, additional examination information, etc.) and/or management information (for example, food intake amount adjustment, target exercise amount, etc.) may be determined as guide information, and the guide information may be provided to the monitoring device. The monitoring device may provide the guide information obtained from the diagnostic device to the subject, and may determine whether the monitored information matches the guide information. As a result of the determination, when matched, the monitoring device may provide information that the subject is observing the guide information well, and when not matched, the monitoring device may provide a warning to the subject to observe the guide information.

In addition, as described in 2.1.5, the processor of the diagnostic device may provide guide information using a predetermined database and/or guide information model. At this time, guide information according to cardiovascular disease diagnostic information and cardiovascular disease progression speed may be matched and stored in the database, or the guide information model may be learned according to guide information according to cardiovascular disease diagnostic information and cardiovascular disease progression speed. Accordingly, the processor of the diagnostic device may obtain guide information from the database and/or guide information model using cardiovascular disease diagnostic information and cardiovascular disease progression speed.

For example, when the QRISK3 score is 8%, which is a low-risk group, and the score of cardiovascular disease diagnostic information is equal to or greater than a predetermined threshold value, the processor of the diagnostic device may determine that the cardiovascular risk of the subject is low, but the cardiovascular disease progression speed of the subject is fast compared to other low-risk groups. Accordingly, the processor of the diagnostic device may provide information that the cardiovascular disease progression speed is fast and/or guide information according thereto (for example, guide information provided when the cardiovascular disease diagnostic information is determined to be moderate risk).

In addition, when the QRISK3 score is 8%, which is a low-risk group, and the score of cardiovascular disease diagnostic information is less than a predetermined threshold value, the processor of the diagnostic device may determine that the cardiovascular risk of the subject is low and the cardiovascular disease progression speed of the subject is slow compared to other low-risk groups. Accordingly, the processor of the diagnostic device may provide information that the cardiovascular disease progression speed is slow and/or guide information according thereto (for example, guide information provided when the cardiovascular disease diagnostic information is determined to be low risk).

In addition, according to embodiments, different threshold values may be applied to cardiovascular disease diagnostic information according to the result value according to the biomarker. For example, the threshold value when QRISK3 is a low-risk group, the threshold value when it is a moderate-risk group, and the threshold value when it is a high-risk group may be set differently in the diagnostic device.

Various embodiments of the present specification may be implemented as software including instructions stored in a machine (e.g., computer)-readable storage medium (Machine-Readable Storage Media). The machine, as a device capable of calling an instruction stored from the storage medium and operating according to the called instruction, may include an electronic device according to the disclosed embodiments. When the instruction is executed by a processor, the processor may perform a function corresponding to the instruction directly, or using other components under the control of the processor. The instruction may include code generated or executed by a compiler or interpreter. The machine-readable storage medium may be provided in the form of a non-transitory storage medium. Here, ‘non-transitory storage medium’ means that it does not include a signal and is tangible, and does not distinguish whether data is stored semi-permanently or temporarily in the storage medium. For example, the ‘non-transitory storage medium’ may include a buffer in which data is temporarily stored.

According to one embodiment, the methods according to various embodiments disclosed in the present specification may be provided included in a Computer Program Product. The computer program product may be traded as a product between a seller and a buyer. The computer program product may be distributed in the form of a machine-readable storage medium (e.g., Compact Disc Read Only Memory, CD-ROM), or online through an application store (e.g., Play Store™). In the case of online distribution, at least a part of the computer program product (e.g., Downloadable App) may be at least temporarily stored in a storage medium such as a manufacturer's server, a server of an application store, or a memory of a relay server, or temporarily generated.

As described above, although the embodiments have been described with limited embodiments and drawings, various modifications and variations are possible from the above description by those of ordinary skill in the art. For example, even if the described technologies are performed in a different order from the described method, and/or the components of the described system, structure, device, circuit, etc. are combined or combined in a form different from the described method, or replaced or substituted by other components or equivalents, appropriate results may be achieved.

Therefore, other implementations, other embodiments, and those equivalent to the claims are also within the scope of the claims described later.

What is claimed is:

1. A control method of a diagnostic device, the method comprising:
obtaining a retinal image of a subject; and
obtaining cardiovascular disease diagnostic information about the subject by using a machine learning model based on the retinal image,
wherein the machine learning model includes a first model and a second model,
wherein the first model is a neural network model, and
wherein the second model is a regression-based machine learning model.

2. The control method of claim 1,
wherein the first model is trained based on first training data—the first training data including a plurality of retinal images—and a result value according to a first biomarker corresponding to the first training data,
wherein the obtaining of the cardiovascular disease diagnostic information about the subject comprises:
inputting the retinal image into the first model to obtain a first score from the first model;
inputting the first score and body information of the subject into the second model to obtain a second score from the second model; and
obtaining the cardiovascular disease diagnostic information based on the second score.

3. The control method of claim 2,
wherein the first biomarker is a Coronary Artery Calcium (CAC) score, and
wherein the first score represents a probability that the coronary artery calcium score of the subject is 0 or greater.

4. The control method of claim 2,
wherein the obtaining of the cardiovascular disease diagnostic information about the subject comprises:
applying a predetermined cutoff value to the second score to obtain, as the cardiovascular disease diagnostic information, any one grade, among a plurality of grades, corresponding to the subject.

5. The control method of claim 4,
wherein the cutoff value is set based on information about a second biomarker at least partially different from the first biomarker.

6. The control method of claim 5,
wherein the cutoff value is set based on a population distribution in groups according to a plurality of grades of the second biomarker.

7. The control method of claim 6,
wherein the second biomarker includes at least one of a PCE score, a QRISK3 score, or a modified Framingham risk score.

8. The control method of claim 4,
wherein the cutoff value is set based on a population distribution in groups according to a plurality of grades classified in accordance with a follow-up observation result of a predetermined population.

9. The control method of claim 1, further comprising:
providing guide information about the subject based on the cardiovascular disease diagnostic information.

10. The control method of claim 9,
wherein the providing of the guide information about the subject based on the cardiovascular disease diagnostic information comprises providing guide information corresponding to the cardiovascular disease diagnostic information by using a pre-stored database or a guide information model.

11. The control method of claim 9,
wherein the providing of the guide information about the subject based on the cardiovascular disease diagnostic information comprises:
obtaining a result value according to a biomarker of the subject; and providing the guide information about the subject by using the result value according to the biomarker and the cardiovascular disease diagnostic information.

12. The control method of claim 11,
wherein, even when the cardiovascular disease diagnostic information is identical, when the result value according to the biomarker of the subject is different, the guide information about the subject differs in accordance with the result value according to the biomarker of the subject.

13. The control method of claim 11,
wherein, when the result value according to the biomarker of the subject is a non-high-risk group and the cardiovascular disease diagnostic information is a high-risk grade, the guide information to be provided in a case where the cardiovascular disease diagnostic information is a high-risk grade is provided.

14. The control method of claim 11,
wherein, when the result value according to the biomarker of the subject is a high-risk group and the cardiovascular disease diagnostic information is a non-high-risk grade, the guide information to be provided in a case where the cardiovascular disease diagnostic information is a non-high-risk grade is provided.

15. The control method of claim 11, further comprising:
determining a progression speed of the cardiovascular disease of the subject by using at least one of the result value according to the biomarker of the subject or past cardiovascular disease diagnostic information of the subject.

16. The control method of claim 15,
wherein the providing of the guide information about the subject based on the cardiovascular disease diagnostic information comprises providing the guide information about the subject by using the progression speed of the cardiovascular disease of the subject and the cardiovascular disease diagnostic information.

17. A non-transitory computer-readable recording medium having a program recorded thereon for executing the method according to claim 1.

18. A diagnostic device, comprising:
a storage module; and
at least one processor,
wherein the at least one processor is configured to:
obtain a retinal image of a subject, and
obtain cardiovascular disease diagnostic information about the subject by using a machine learning model stored in the storage module based on the retinal image,
wherein the machine learning model includes a first model and a second model,
wherein the first model is a neural network model, and
wherein the second model is a regression-based machine learning model.
